(19) United States
(12) Patent Application Publication
(10) Pub. No.: US 2026/0348836 A1
(43) Pub. Date: Nov. 5, 2026

(54) POLARIZATION MICROSCOPY BASED CHARACTERIZATION OF BIREFRINGENT FEATURES

(71) Applicant: Regents of the University of Minnesota, Minneapolis, MN (US)

(72) Inventors: Paolo Provenzano, St Paul, MN (US); Guhan Qian, MINNEAPOLIS, MN (US)

(21) Appl. No.: 19/668,069

(22) Filed: May 5, 2026

Related U.S. Application Data
(60) Provisional application No. 63/799,987, filed on May 5, 2025.

(51) Int. Cl. G01N 21/23; G01N 21/17; G01N 33/575; G01N 33/68

(52) U.S. Cl. CPC G01N 21/23; G01N 33/5758; G01N 33/68; G01N 2021/1772; G01N 2021/1776

(57) ABSTRACT

A system for characterizing birefringent features includes processing circuitry configured to: receive image data that includes optically coded spatial and angular information about one or more birefringent features; apply at least one of a phase correction or an amplitude correction to resolve at least one of a hue distortion or an amplitude distortion affecting the optically coded spatial and angular information to generate corrected spatial and angular information; generate, based on the corrected spatial and angular information, information that characterizes the one or more birefringent features; and output the information that characterizes the one or more birefringent features.

### GOVERNMENT RIGHTS AND GOVERNMENT AGENCY SUPPORT NOTICE

This invention was made with government support under CA254849, and CA268069 awarded by the National Institutes of Health. The government has certain rights in the invention.

This application claims the benefit of U.S. Provisional Patent Application No. 63/799,987, filed May 5, 2025, and titled “POLARIZATION MICROSCOPY BASED CHARACTERIZATION OF BIREFRINGENT FEATURES,” the entire contents of which are incorporated herein by reference.

### TECHNICAL FIELD

The present disclosure relates to systems and methods for characterizing birefringent features based on polarization microscopy techniques.

### BACKGROUND

Being able to characterize birefringent features can be useful in diagnostic and preventative care applications, among others. For example, pancreatic ductal adenocarcinoma (PDA) and many other solid tumors may include birefringent collagen fiber patterns known as tumor-associated collagen signatures (TACS), which have been shown to either limit or direct cell migration into and through the stroma. Included in the TACS panel are TACS-2 and -3 that represent matrix fibers aligned parallel to either ductal structures or carcinoma clusters within the tumor mass, or fibers aligned perpendicular to these structures, respectively. Furthermore, in advanced and poorly differentiated disease where single or small clusters of carcinoma cells are spread throughout the extracellular matrix (ECM), these aligned TACS-3 architectures serve to continue to direct cell migration through the tumor mass via contact guidance. TACS architectures are established as drivers of invasion and metastasis, have prognostic value for patient outcomes, and are valuable in augmenting pathologic disease assessment in live or archival tumor samples.

### SUMMARY

This disclosure describes systems and methods for characterizing birefringent features, such as tumor-associated collagen signatures (TACS), other extracellular matrix (ECM) structures, and other organic or inorganic birefringent materials, based on polarization microscopy techniques. Polarization microscopy techniques, such as polychromatic polarization microscopy (PPM), can provide images that contain optically coded (e.g., color and/or intensity coded) spatial and angular information about birefringent features. However, polarization microscopy images may have hue distortions resulting from histological staining, such as Hematoxylin and Eosin (H&E) staining, or any other type of preparatory staining applied to specimen slides. Hue can also be affected by ambient lighting, e.g., in a laboratory or office space where microscopy is being performed. Polarization microscopy images may also have amplitude distortions due to non-uniform spectral response of an imaging sensor or another contributing factor. This disclosure describes polarization microscopy-based characterization techniques that apply phase and/or amplitude correction to resolve distortions. In this manner, the techniques of this disclosure may leverage the optically coded spatial and angular information contained in polarization microscopy images to provide advanced characterizations of imaged birefringent features based on quantification and/or spatial analysis of those features.

In an example, a system for characterizing birefringent features includes processing circuitry configured to: receive image data that includes optically coded spatial and angular information about one or more birefringent features; apply at least one of a phase correction or an amplitude correction to resolve at least one of a hue distortion or an amplitude distortion affecting the optically coded spatial and angular information to generate corrected spatial and angular information; generate, based on the corrected spatial and angular information, information that characterizes the one or more birefringent features; and output the information that characterizes the one or more birefringent features.

In another example, a method of characterizing birefringent features includes: receiving, by processing circuitry, image data that includes optically coded spatial and angular information about one or more birefringent features; applying, by the processing circuitry, at least one of a phase correction or an amplitude correction to resolve at least one of a hue distortion or an amplitude distortion affecting the optically coded spatial and angular information to generate corrected spatial and angular information; generating, by the processing circuitry, based on the corrected spatial and angular information, information that characterizes the one or more birefringent features; and outputting, by the processing circuitry, the information that characterizes the one or more birefringent features.

In another example, a non-transitory computer-readable storage medium includes instructions encoded thereon, the instructions configured to cause processing circuitry to: receive image data that includes optically coded spatial and angular information about one or more birefringent features; apply at least one of a phase correction or an amplitude correction to resolve at least one of a hue distortion or an amplitude distortion affecting the optically coded spatial and angular information to generate corrected spatial and angular information; generate, based on the corrected spatial and angular information, information that characterizes the one or more birefringent features; and output the information that characterizes the one or more birefringent features.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1A is a block diagram of an example system for characterizing birefringent features based on polarization microscopy.

FIG. 1B is a schematic illustrating an example configuration of a polarization microscopy system.

FIG. 2 is a flow diagram of an example process for characterizing birefringent features based on polarization microscopy.

FIG. 3 is an example pattern of a calibration slide for the polarization microscopy system.

FIG. 4 is an example of images taken by second-harmonic generation (SHG) microscopy, bright-field (BF) microscopy, and polarization microscopy systems.

FIG. 5 is a flow diagram of an example process for performing feature segmentation on image data.

FIG. 6A is an example of an image collected by a polarization microscopy system.

FIG. 6B is an example of a binary classification map based on the image of FIG. 6A.

FIG. 6C is an example of a segmented image of birefringent features of the image of FIG. 6A.

FIG. 7A is an example of mask patterns for image calibration.

FIG. 7B is an example of mask patterns for image calibration.

FIG. 7C is an example plot illustrating a correction applied to image data.

FIG. 7D is an example plot illustrating a correction applied to image data.

FIG. 8 is an example plot illustrating a correction curve.

FIG. 9A is an example plot of corrected and uncorrected image data.

FIG. 9B is an example plot of corrected and uncorrected image data.

FIG. 10 is an example plot of a polynomial fitting of a hue histogram of the image data.

FIG. 11A is an example image with an identified region of interest (ROI).

FIG. 11B is an example image with an identified ROI.

FIG. 12A is an example plot of corrected and uncorrected image data for the ROI of FIG. 11A.

FIG. 12B is an example plot of corrected and uncorrected image data for the ROI of FIG. 11B.

FIG. 13 is an example of image data illustrating pancreatic ductal adenocarcinoma (PDA) disease progression over time.

FIG. 14 is an example of angular information plots associated with the image data in FIG. 13.

FIGS. 15A and 15B are examples of spatial information plots associated with the image data in FIG. 13.

FIG. 16 is a conceptual diagram of an example pixel-wise analysis to extract information about birefringent features, such as tumor-associated collagen signatures (TACS), from image data.

FIG. 17 is a flow diagram illustrating an example of various stages of PDA disease progression.

FIGS. 18A-18E are example images of various stages of PDA disease progression.

FIGS. 19A-19E are example plots corresponding to the example images of FIGS. 18A-18E, respectively.

FIG. 20 is an example plot of TACS-3 clusters at various stages of PDA disease progression.

FIG. 21 is an example plot of TACS scores at dissemination edges and non-dissemination edges of an imaged sample.

FIG. 22 is a conceptual diagram illustrating an example of extracellular matrix (ECM) architecture in metastatic PDA lesions.

### DETAILED DESCRIPTION

This disclosure describes systems and methods for characterizing birefringent features, such as tumor-associated collagen signatures (TACS), based on polarization microscopy techniques. In some examples, the techniques of this disclosure may be applied to diagnostic testing or analysis of tissue samples to assess onset or progression of pancreatic ductal adenocarcinoma (PDA) or pancreatic intraepithelial neoplasia (PanINs), a well-known precursor to PDA. Detection and characterization of TACS involves capturing TACS aligned extracellular matrix (ECM) structures that are comprised of birefringent material. However, the techniques may be applied to polarization microscopy signals associated with any birefringent organic materials (e.g., to characterize wound healing, connective tissues, etc.) or inorganic materials (e.g., sedimentary structures). In general, the techniques of this disclosure may be applied to detect and characterize birefringent features in organic material and inorganic material samples.

PDA often presents as advanced disease with a poor resectability rate, aggressive metastatic spread, and limited effective treatment options. This is due in part to PDA predominantly associated with a robust fibroinflammatory response with hyperactivated stromal fibroblasts, extensive immunosuppression, and vastly elevated ECM deposition. In addition to limiting the distribution and efficacy of both chemotherapy and immunotherapy, the ECM contributes to the aggressive metastatic spread observed in PDA, including early dissemination. PDA and many other solid tumors possess defined collagen fiber patterns known as TACS, which have been shown to either limit or direct cell migration into and through the stroma. Included in the TACS panel are TACS-2 and -3 that represent matrix fibers aligned parallel to either ductal structures or carcinoma clusters within the tumor mass, or fibers aligned perpendicular to these structures, respectively. Furthermore, in advanced and poorly differentiated disease where single or small clusters of carcinoma cells are spread throughout the ECM, these aligned TACS-3 architectures serve to continue to direct cell migration through the tumor mass via contact guidance. These architectures are established as drivers of invasion and metastasis, have prognostic value for patient outcomes, and are valuable in augmenting pathologic disease assessment in live or archival tumor samples.

One imaging technique for evaluating and characterizing collagen fiber matrices is second-harmonic generation (SHG) microscopy, a nonlinear optical imaging technique that exploits the non-centrosymmetric structure and second-order nonlinear susceptibility of collagen to produce bright and specific contrast without labeling. While SHG is an effective approach, particularly for analysis within 3D environments and live tissue/tumor systems, that can provide quantitative structural information, access to SHG can be limited due to the high cost of high-power femtosecond pulsed lasers and associated microscope instrumentation, particularly in clinical and pathologic assessment settings. As an alternative, staining techniques (e.g., based on Masson's trichrome stain, Movat's pentachrome stain, or picrosirius red stain) are sometimes employed to image collagen in fixed thin pathology sections. However, these sometimes result in dense staining regions that obscure fiber edges and when imaged do not directly provide angular information. In addition, the stain-based imaging approaches may involve prolonged preparation processes, which can be additionally problematic if rapid turnover times are needed.

Polarization microscopy techniques, such as PPM, may provide a convenient and low-cost addition to a traditional light microscope in order to observe ECM architecture in un-stained tissue or in classic H&E stained samples, where fundamental cellular phenotypes for grading are readily observable, by providing orientation-dependent full spectrum colors. Thus, polarization microscopy offers a unique opportunity in collagen-based research and clinical diagnosis that sheds light on collagen fiber architectures that drive disease progression, including early dissemination architectures.

However, polarization microscopy images may have hue distortions resulting from H&E staining, or any other type of preparatory staining applied to specimen slides. To address hue distortions and other barriers to the effective use of polarization microscopy for characterization of birefringent features, this disclosure describes a computational analysis workflow for fiber segmentation, which, in some examples, may use machine learning (ML), followed by a correction function for color-angle hue shift and intensity correction and a pixel-wise TACS characterization algorithm that computes TACS regression scores relative to a tunable boundary. Using this optical analysis suite, it is possible to robustly capture target TACS architectures in both early and late-stage disease. The techniques of this disclosure may be applied to faithfully capture TACS architectures that drive early dissemination in PDA, thus allowing for quantitative analysis of histology sections. Furthermore, the techniques of this disclosure may be applied to examine TACS architectures in native metastatic lesions and identify paths of focal invasion and dissemination as well as new structural information regarding anchoring of TACS into normal adjacent tissue. The techniques of this disclosure may allow polarization microscopy to be used as a powerful tool for analyzing ECM architectures in tumors that can be employed to define patterns with clinical significance.

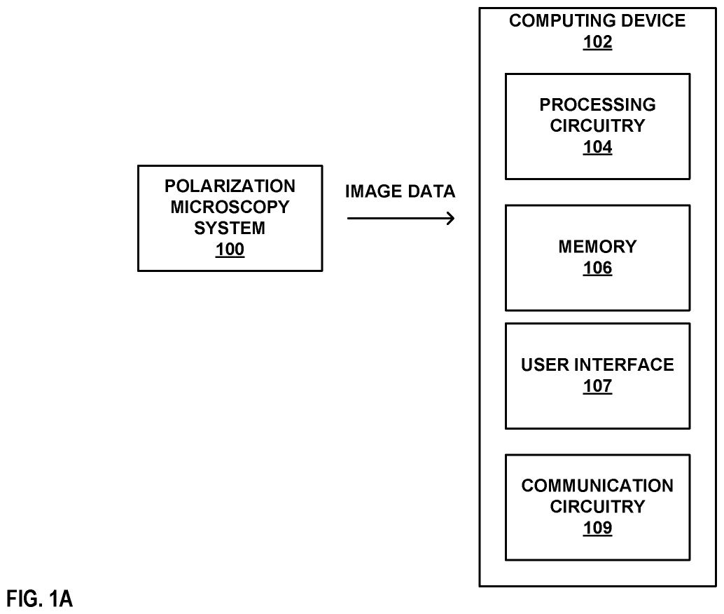

FIG. 1A is a block diagram of an example system for characterizing birefringent features, such as TACS, based on polarization microscopy. In the example of FIG. 1, the system includes a polarization microscopy system 100 (e.g., a PPM system) and a computing device 102. The computing device 102 may be configured to receive image data (e.g., images) collected by polarization microscopy system 100. In some examples, the computing device 102 is communicatively coupled to polarization microscopy system 100 via a wired or wireless communication link. In other examples, the computing device 102 may be included in polarization microscopy system 100, or within a polarization microscopy attachment that is configured to be coupled to a microscope (e.g., a standard off-the-shelf microscope) to form polarization microscopy system 100.

Computing device 102 may include processing circuitry 104 (e.g., one or more microprocessors, microcontrollers, or the like) and memory 106. Processing circuitry 104 may be configured to execute program instructions from memory 106 to perform any functions, described herein, of computing device 102, processing circuitry 104, and/or polarization microscopy system 100.

FIG. 1B is a schematic illustrating an example configuration of polarization microscopy system 100. In the example of FIG. 1B, polarization microscopy system 100 includes a light source 108, a polarization state generator 110, a condenser lens 112, a slide holder 114 configured to hold a specimen slide (e.g., H&E slide), an objective lens 116, a polarization state analyzer 118, and an image sensor 120. In some examples, one or more of these components, such as polarization state generator 110 and/or polarization state analyzer 118, may be included in a polarization microscopy attachment that is configured to be coupled to a microscope to form polarization microscopy system 100.

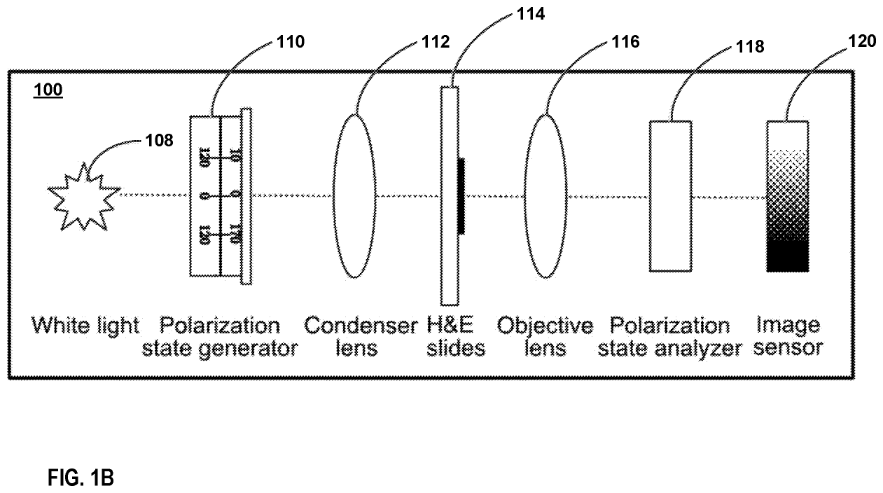

FIG. 2 is a flow diagram illustrating an example process that may be executed by processing circuitry 104 to characterize birefringent features in image data that includes optically coded spatial and angular information about one or more birefringent features, such as image data collected via polarization microscopy system 100.

In the example of FIG. 2, processing circuitry 104 receives image data that includes optically coded spatial and angular information about one or more birefringent features (200). In some examples, the image data includes optically coded spatial and angular information about one or more birefringent features (e.g., TACS).

In some examples, processing circuitry 104 may perform feature segmentation on the image data to generate segmented image data (202). Performing feature segmentation may separate targeted birefringent features, such as collagen fibers, from non-birefringent background components. Separating the targeted birefringent features may address segmentation challenges caused by hue similarities between polarization microscopy signals and preparatory stains, as well as heterogeneity in staining density. To perform the feature segmentation, processing circuitry 104 may apply a machine learning classifier to the image data.

To generate the machine learning classifier, processing circuitry 104 may receive labeled training images containing annotations identifying the targeted birefringent features and annotations identifying the background components. Processing circuitry 104 may execute a random trees algorithm or a similar machine learning algorithm using the labeled training images to train the machine learning classifier. Processing circuitry 104 may execute the trained machine learning classifier on the image data to generate a classification map. The classification map may identify pixels within the image data as either the targeted birefringent features or the background components.

Processing circuitry 104 may utilize the classification map as a binary mask. Processing circuitry 104 may apply the binary mask to the image data to isolate the targeted birefringent features. Applying the binary mask may yield the segmented image data. The segmented image data may isolate the optically coded spatial and angular information of the targeted birefringent features for subsequent phase correction, amplitude correction, and pixel-wise analysis operations.

Processing circuitry 104 may apply a phase and/or amplitude correction to resolve a hue and/or amplitude distortion affecting the optically coded spatial and angular information in order to generate corrected spatial and angular information (204). Histological staining or ambient lighting conditions may shift the hue of the birefringent features. Non-uniform spectral responses of an image sensor may cause amplitude distortions. Resolving the hue distortion or the amplitude distortion may align an angle axis to a hue axis and may normalize intensity peaks to provide accurate angular measurements. In some examples, processing circuitry 104 is configured to apply the phase and/or amplitude correction to segmented image data.

To apply an amplitude correction, processing circuitry 104 may generate a hue correction curve. Generating the hue correction curve may include filtering a hue channel of the image data using a Gaussian filter or a median filter to remove optical artifacts. Processing circuitry 104 may detect peaks in a hue histogram corresponding to distinct angles and may apply modified Akima interpolation to extract an envelope of the hue histogram as a function of intensity. Processing circuitry 104 may sample the hue correction curve to obtain a normalization multiplier and may apply the normalization multiplier to the image data to normalize the amplitude distortion.

To apply a phase correction, processing circuitry 104 may calculate a corrective phase shift representing a difference between a measured angle and a reference angle. Calculating the corrective phase shift may include converting the image data into a hue saturation value color space and detecting a global maximum peak within a hue histogram to identify the measured angle. Processing circuitry 104 may apply a circular shift to an uncorrected hue angle based on the corrective phase shift to resolve the hue distortion.

Processing circuitry 104 may also determine and apply the phase correction or the amplitude correction utilizing alternative techniques of the disclosure. Processing circuitry 104 may apply a predefined correction profile stored in memory corresponding to known spectral response variations of the image sensor. Processing circuitry 104 may apply algorithmic estimations to correct the hue distortion or the amplitude distortion by extracting baseline hue values from regions of the image data known to lack birefringent features. In some examples, processing circuitry 104 may execute machine learning models trained on paired datasets of distorted and calibrated polarization microscopy images to predict and apply the phase correction or the amplitude correction directly to the image data.

In some examples, processing circuitry 104 may perform a pixel-wise analysis on the image data to extract spatial and angular information about the one or more birefringent features from the corrected spatial and angular information to generate extracted spatial and angular information (206). Processing circuitry 104 may be configured to perform the pixel-wise analysis on segmented image data to extract the spatial and angular information about the one or more birefringent features from the corrected spatial and angular information.

Performing the pixel-wise analysis may include computing spatial regression scores relative to a boundary of a region of interest. For example, to perform the pixel-wise analysis, processing circuitry 104 may receive a selection of a first region of interest defining a contour boundary and a second region of interest identifying associated birefringent features. Processing circuitry 104 may calculate normal vectors orthogonal to contour pixels located along the contour boundary of the first region of interest.

Processing circuitry 104 may identify intersecting fiber pixels associated with the birefringent features. Processing circuitry 104 may calculate a dot product for the intersecting fiber pixels to determine an angle between the intersecting fiber pixels and the normal vectors. Processing circuitry 104 may apply a weighting function to the intersecting fiber pixels to mitigate distance bias. Applying the weighting function may include evaluating the intersecting fiber pixels on a Gaussian curve such that intersecting fiber pixels located closer to the contour boundary receive larger weights and intersecting fiber pixels located further from the contour boundary receive smaller weights.

Processing circuitry 104 may compute a weighted average of the angles to generate a regression score for the contour pixels. Generating the extracted spatial and angular information may include transforming the weighted average of the angles onto a defined scale to classify the birefringent features. Processing circuitry 104 may map the regression score to a visual classification overlay to generate spatial distribution maps characterizing the birefringent features relative to the first region of interest.

Processing circuitry 104 may generate, based on the corrected spatial and angular information, information that characterizes the one or more birefringent features (208). Generating the information that characterizes the one or more birefringent features may include determining a quantification or a spatial distribution of specific collagen architectures present in the image data. In some examples, processing circuitry 104 may generate the information that characterizes the one or more birefringent features based on the extracted spatial and angular information resulting from a pixel-wise analysis. The extracted spatial and angular information may be utilized to compute regression scores classifying the birefringent features into distinct signature categories, such as distinguishing parallel tumor-associated collagen signatures from perpendicular tumor-associated collagen signatures.

The information that characterizes the one or more birefringent features may encompass a variety of metrics and data types. For example, processing circuitry 104 may extract morphological metrics including collagen fiber length, width, and straightness utilizing curvelet transform extraction algorithms. In some examples, processing circuitry 104 may generate density measurements, localized alignment scoring, feature boundary mapping, structural integrity estimations, and algorithmic classification labels. In some examples, processing circuitry 104 may apply clustering algorithms to the corrected spatial and angular information to group the birefringent features into spatial distribution maps. In some examples, processing circuitry 104 may output quantification metrics defining a total area, a total volume, or a relative proportion of the birefringent features located within a selected region of interest.

In some examples, processing circuitry 104 may generate diagnostic information based on the quantification and the spatial distribution of the birefringent features. Generating the diagnostic information may include mapping a computed regression score to a visual scale for evaluation of disease progression or tissue remodeling. The diagnostic information may indicate transition stages of early cellular dissemination, localized tissue deformation, or degrees of focal invasion into adjacent tissue structures.

As discussed in the examples provided below, the one or more birefringent features may include TACS or other collagen fiber patterns. In some examples, to generate the information that characterizes the one or more birefringent features, processing circuitry 104 is configured to determine at least one of a quantification or a spatial distribution of one or more types of TACS present in the image data. Processing circuitry 104 may also generate diagnostic information (e.g., the information that characterizes the one or more birefringent features, or at least a portion thereof) based on the quantification and/or spatial distribution of the one or more types of TACS present in the image data. In some examples, processing circuitry 104 is further configured to receive a user input indicating a region of interest in the image data. Processing circuitry 104 may be configured to limit the information that characterizes the one or more birefringent features to the region of interest. In some examples, processing circuitry 104 can also limit one or more of the image processing and/or analysis steps (e.g., feature segmentation, phase correction, and/or pixel-wise analysis) to the region of interest to improve processing efficiency (e.g., save time, power, or processor utilization).

Processing circuitry may be further configured to output the information that characterizes the one or more birefringent features. For example, processing circuitry 104 may be configured to output the information that characterizes the one or more birefringent features via a user interface 107, such as a display, a projector, a speaker, a printer, or any other output device. Additionally or alternatively, processing circuitry 104 may be configured to transmit the information that characterizes the one or more birefringent features to another device. For example, processing circuitry 104 may transmit the information via communication circuitry 109, such as a wired or wireless transmitter, transceiver, or the like. Communication circuitry 109 may be configured to operate over any communication protocol, including, but not limited to, a Universal Serial Bus (USB) interface, a Thunderbolt interface, an Ethernet interface, a local area network (LAN), a wireless LAN (e.g., a Wi-Fi® network), a cellular network, a near-field communication (NFC) interface, and/or a personal area network (PAN) (e.g., a Bluetooth® connection).

In some examples, feature segmentation and pixel-wise analysis operations (i.e., operations 202 and 206, described above) are optional steps, as indicated by dashed lines in FIG. 2. Processing circuitry 104 may be configured to perform one, both, or none of these operations when executing the method that is illustrated in FIG. 2.

Example implementations of the system of FIGS. 1A-1B and the methodology of FIG. 2 are described below in the context of using PPM to detect and characterize collagen architectures associated with PDA. However, the computational methodologies can be applied to any other type of polarization microscopy system 100 and to analyze any other type of birefringent feature that may be present in an organic material or inorganic material sample.

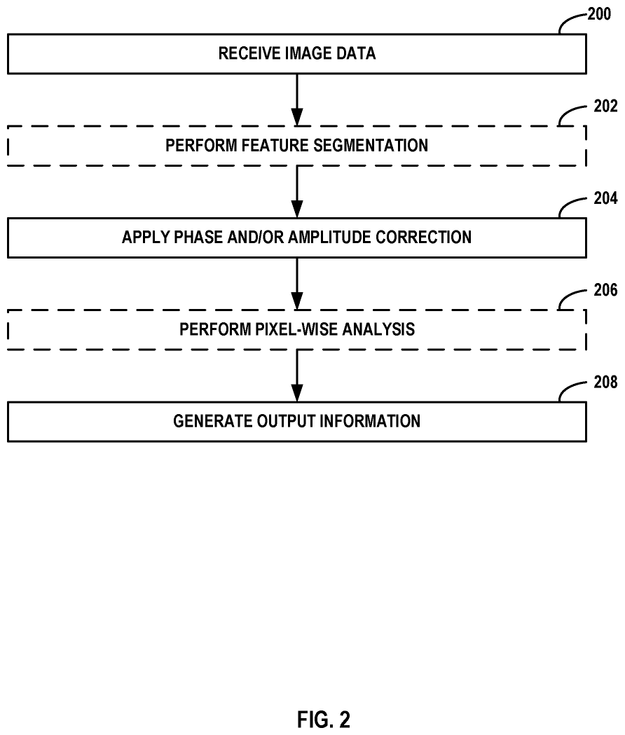

In the examples described below, PPM reveals dense collagen architectures in autochthonous PDA with inherent angular information and provides label-free collagen contrast with ECM information on a benchtop. FIG. 3 is an example of a calibration slide used to implement PPM based techniques to characterize collagen architectures, e.g., TACS. In the example of FIG. 3, the calibration slide contains two-part birefringent patterns imprinted on a silica cover glass. The star pattern (left) comprises three segments of varying linewidth at d=20 μm (1-line), 40 μm (3-line), and 60 μm (5-line), with each wedge separated by π/16 radian. Three 20 μm×20 μm perpendicular linear gratings (right) have increasing line densities from left to right at 350, 550, and 1050 lp/mm.

In the example of FIG. 1B, the optical schematic of polarization microscopy system 100 (e.g., a PPM system) shows light source 108 (e.g., a white light source) passing through polarization state generator 110 (e.g., z-cut quartz), resulting in a wavelength-dependent rotation of polarization states. After introducing additional birefringence in the ECM presented in PDA and metastatic PDA samples, the unaltered polarization states are distinguished with polarization state analyzer 118 by reversing the polarization rotation, resulting in Newton colors dependent on collagen fiber orientation.

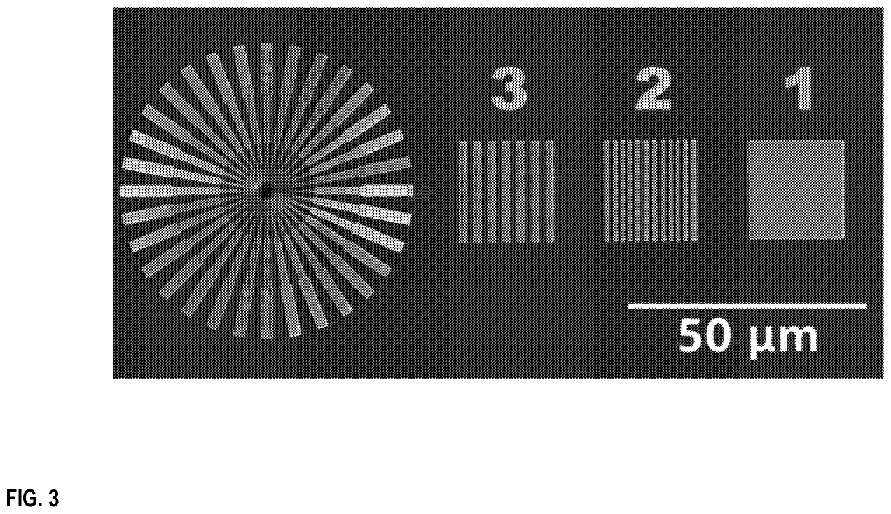

As shown in the example of FIG. 4, PPM demonstrates comparable collagen sensitivity to SHG imaging in thin 2D sections. In this example, the same location in an H&E slide of primary tumor from Kras^{G12D/+}; Tp53^{R172H/+}; Pdx1-Cre (KPC) mice was imaged with SHG, BF, PPM 20X, and PPM 40X. PPM produces bright contrast of the ECM and reveals detailed angular ECM information by producing analog hue gradients on birefringent materials, which can be directly examined through the eyepiece or digitally captured by image sensors. In contrast, SHG delivers excellent collagen contrast through nonlinear optics but lacks inherent angular information about the ECM. In BF microscopy, the collagen contrast is mostly absent.

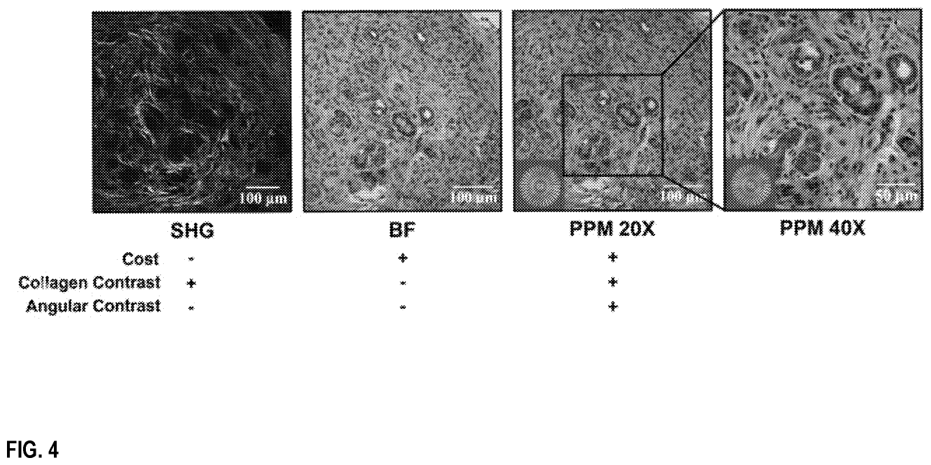

FIG. 5 is a flow diagram illustrating a machine learning-based collagen segmentation and calibration tool (e.g., software executed by processing circuitry 104), referred to herein as PPM Image Kalibrator (PIKL), that produces accurate hue-based collagen metrics. In the example of FIG. 5, collagen fibers may be segmented with a feature segmentation tool (e.g., software executed by processing circuitry 104), such as QuPath: Open source software for digital pathology image analysis, using a five-step workflow with a machine learning classifier, which allows more degrees of freedom in tunability and generalizability across various tissue samples. In some examples, the five-step workflow includes image analysis operations comprising fiber labeling 300, classifier training 302, ML inference 304, performing a quality check 306, and repeating operations 300-306 as needed based on the quality check 306, followed by one or more segmentation operations (e.g., fiber segmentation 308).

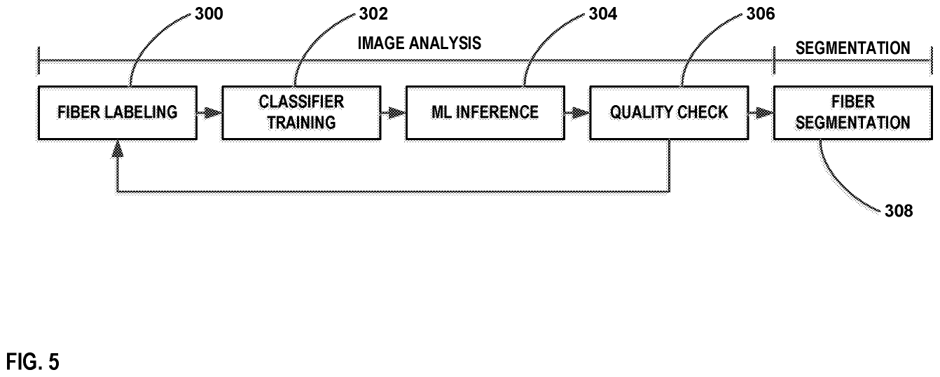

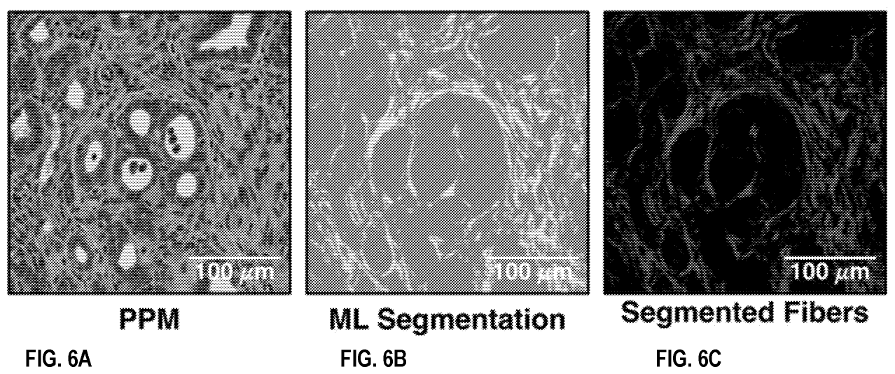

As shown in the example of FIGS. 6A-6C, a PPM image (FIG. 6A) may be segmented with the proposed workflow. A binary classification map (FIG. 6B) is then generated, and the corresponding fiber-only image (FIG. 6C) may then be obtained through masking. As demonstrated in FIGS. 7A-7B, angle and hue are inherently circular and periodic. Linear patterns at π/2 radian from the +x axis may exhibit a phase shift with respect to their ground truth angle. FIGS. 7C and 7D illustrate an example of how PIKL may correct the phase shift for all hue-angle pairs to facilitate downstream analysis that necessitates continuity for hue histogram values as a function for all directions α∈[0, π]. In this example, the star pattern was used to calibrate the non-uniform spectral response in the sensor during image acquisition.

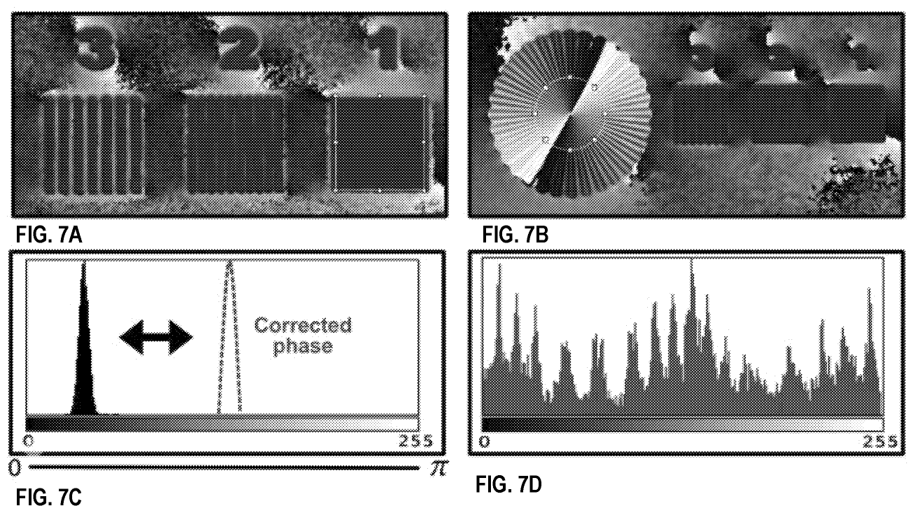

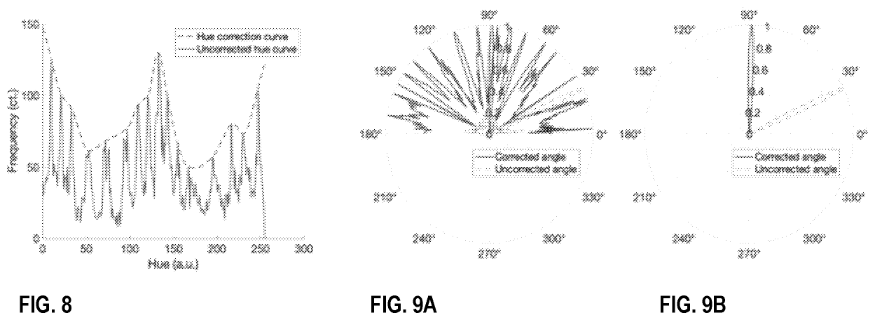

FIG. 8 illustrates a correction curve that may be obtained to calibrate the amplitude of all hue histogram values. Using the calibration slide, PIKL can be applied as demonstrated in corrected polar plots (solid line plots) in contrast to the uncorrected polar plots (dashed line plots) of FIGS. 9A and 9B. In an example, the corrected histogram for the star pattern exhibits 16 distinct peaks with uniform amplitudes, whereas the uncorrected plot erroneously displays indistinguishable peaks. Furthermore, the phase shift correction implemented in PIKL may yield accurate angular measurement, as demonstrated by the phase rotation of the multiplet produced from the star pattern and the singlet from the linear pattern.

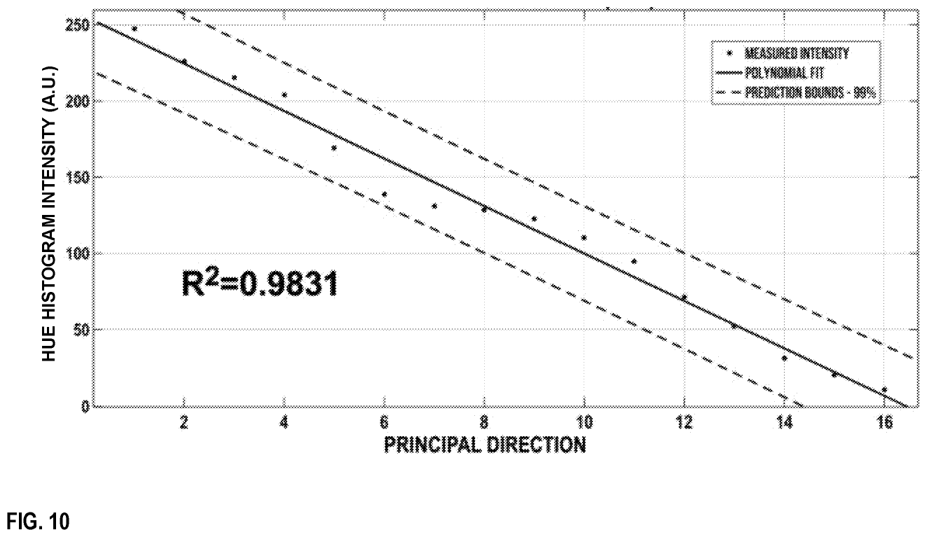

FIG. 10 illustrates a polynomial fitting of the hue histogram value as a function of direction indicated a high goodness of fit (R2=0.9831), enabling angle measurement by directly evaluating the polynomial with an arbitrary hue value.

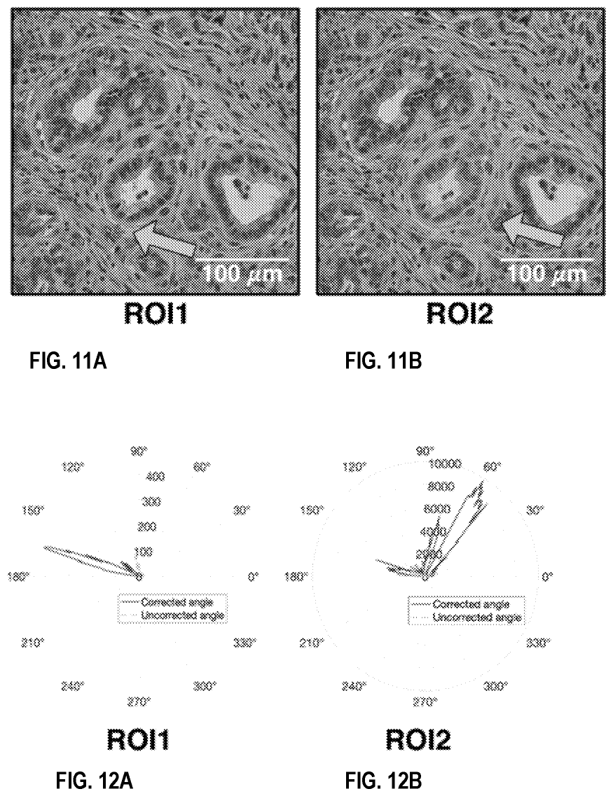

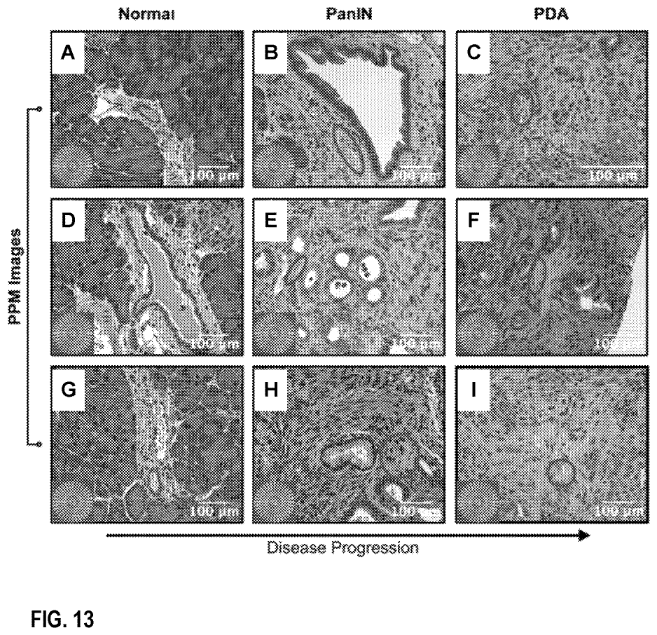

FIGS. 11A and 11B illustrate two ROIs selected to evaluate PIKL calibration with polar plots. In this example, a relatively smaller ROI (FIG. 11A, arrow) within a highly aligned collagen fiber bundle produces a prominent narrow peak at 165.5° (FIG. 12A), while a relatively larger ROI (FIG. 11B, arrow) encompassing an entire pancreas duct and ECM produces a broad peak at 55.5° (FIG. 12B) and several other narrow peaks, reflecting the overall fiber orientations within the user-defined ROI.

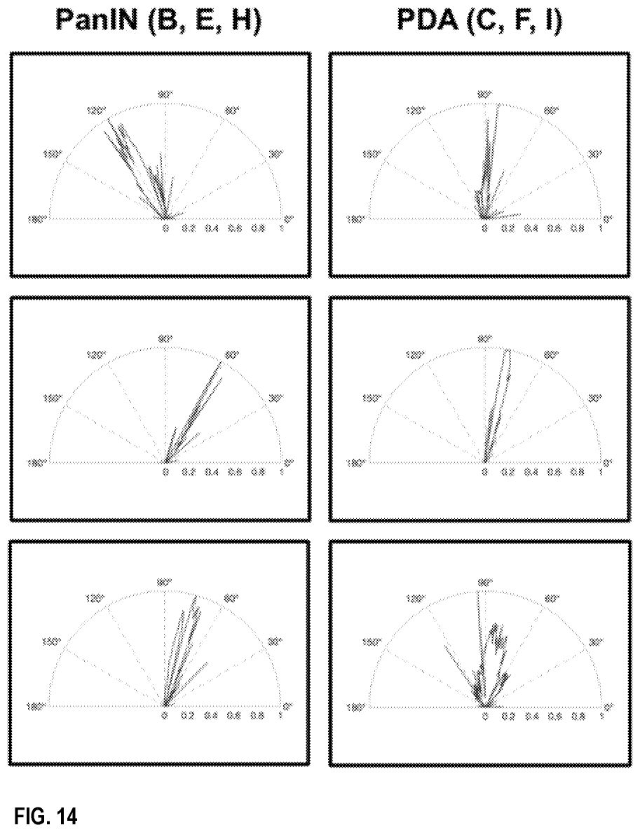

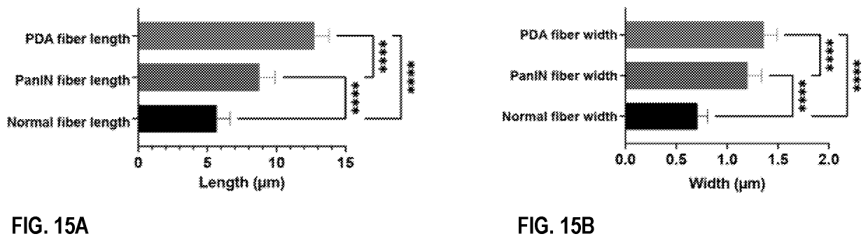

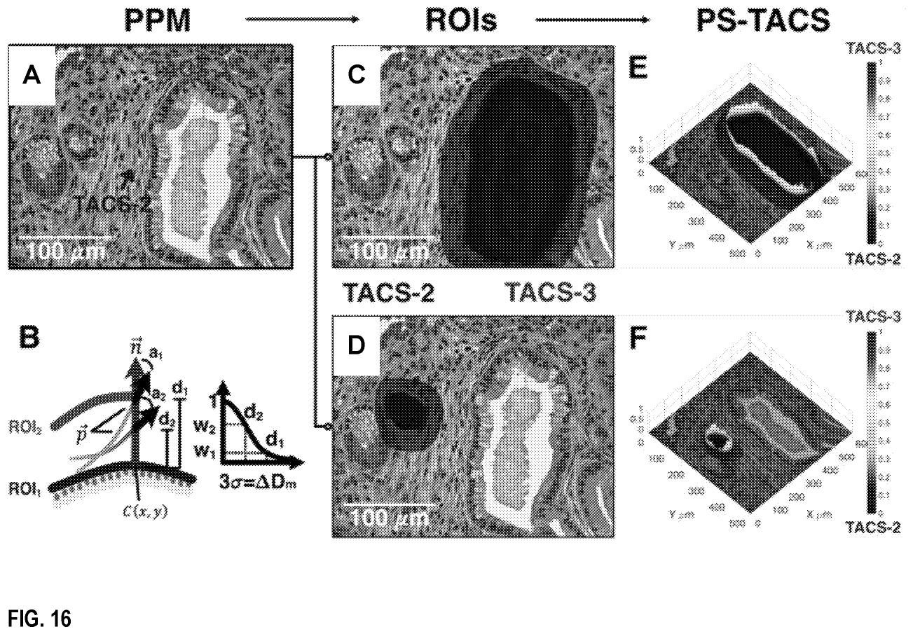

FIGS. 13-15B demonstrates an example of how quantitative analysis of PPM images captures collagen architectures associated with PDA progression. In the example of FIG. 13, images A, D, and G are PPM images of normal pancreas H&E slides with visible pancreas ducts, acinar 14cells, and islets. In the example of FIG. 13, images B, E, and H are PPM images of early-stage disease, where PanINs are present with robust desmoplastic ECM. Collagen morphology is notably heterogeneous and diverse, which is demonstrated by the meandering architecture in FIG. 13, image B, and large aligned collagen bundles in FIG. 13, image H. In this example, collagen length and width are increased, and TACS-3 hotspots are present that primes for dissemination through aberrant basal extrusion. In the example of FIG. 13, images C, F, and I are PPM images of PDA, wherein robust aligned collagen is present with carcinoma cells that are robustly invasive, using TACS-3 hotspots for contact guidance to drive dissemination. Angles of circled regions in FIG. 13: B, C, E, F, H, and I are shown FIG. 14. In the example of FIGS. 15A and 15B, collagen fiber length and width are quantified with quantification tool (e.g., software executed by processing circuitry 104), such as curvelet transform fiber extraction (CT-FIRE), a software tool developed at the Laboratory for Optical and Computational Instrumentation (LOCI), University of Wisconsin-Madison, for automatically extracting and quantifying individual collagen fibers from microscopic images. FIGS. 15A and 15B show progressively longer (****p<0.0001) and thicker (****p<0.0001) fibers with advanced disease progression (one-way ANOVA w/post hoc Tukey test).

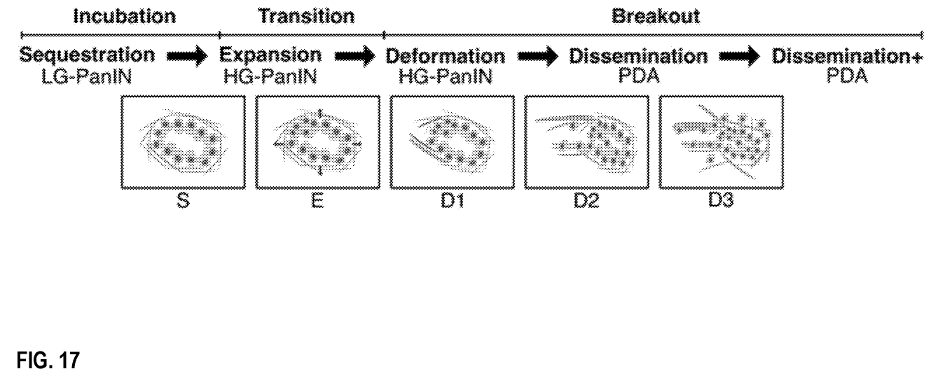

FIG. 16 is a flow diagram illustrating an example process implemented by a pixel-wise analysis tool (e.g., software executed by processing circuitry 104), referred to herein as Polychromatic Spatial TACS (PS-TACS), that computes pixel-wise spatial TACS regression. In the example of FIG. 16, prominent PanIN with mixed TACS highlights the heterogeneity in stromal collagen architecture, emphasizing the utility for spatially resolved TACS computation (FIG. 16: A). PS-TACS iterates through all pixels along ROI₁ boundary and computes their normal vectors (FIG. 16: B). Fiber pixels intersected by the normal vector form a tangent vector, and the angles between each fiber pixel and the normal vector are determined by calculating the dot products. To mitigate distance bias, fiber pixels along the normal vector may be evaluated on a Gaussian function, such that closer fibers have larger weights in the determination of TACS. The TACS score of that pixel is thus the weighted average of angles transformed on a scale of [−1, 1]. PS-TACS enables users to define various ROIs within a PPM image (FIG. 16: C-D). For example, pancreatic ducts and ECM may be selected by the user, and binary TACS classification may be calculated and overlaid on the ductal contour. Pixel-wise TACS regression is then displayed in a 3D surface plot and overlaid on the PPM image (FIG. 16: E-F). In some examples, a TACS score is mapped to a linear scale from 0 (TACS-2) to 1 (TACS-3) for visualization.

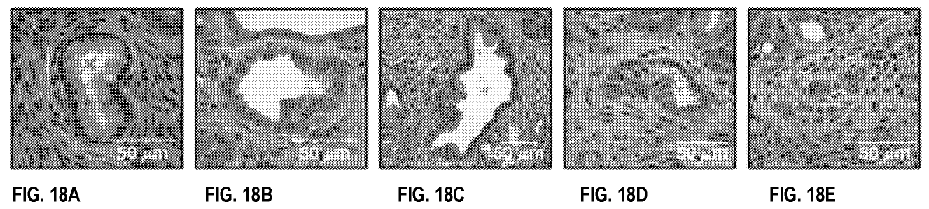

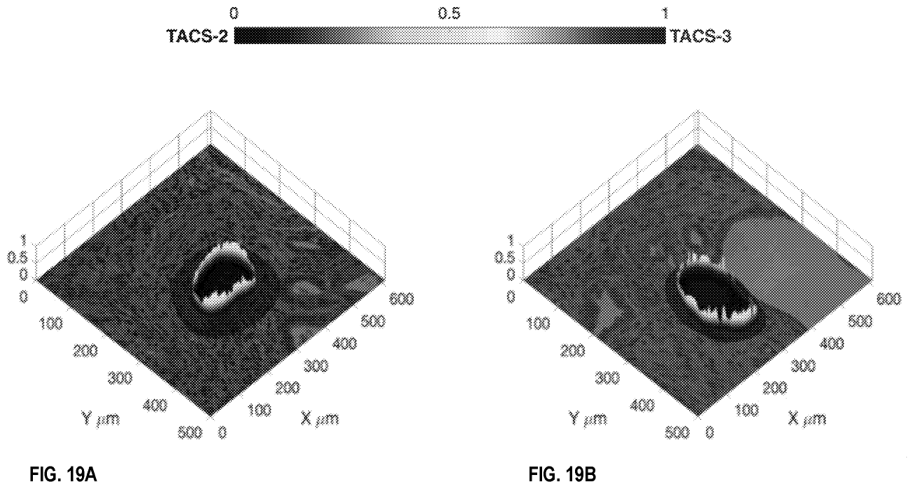

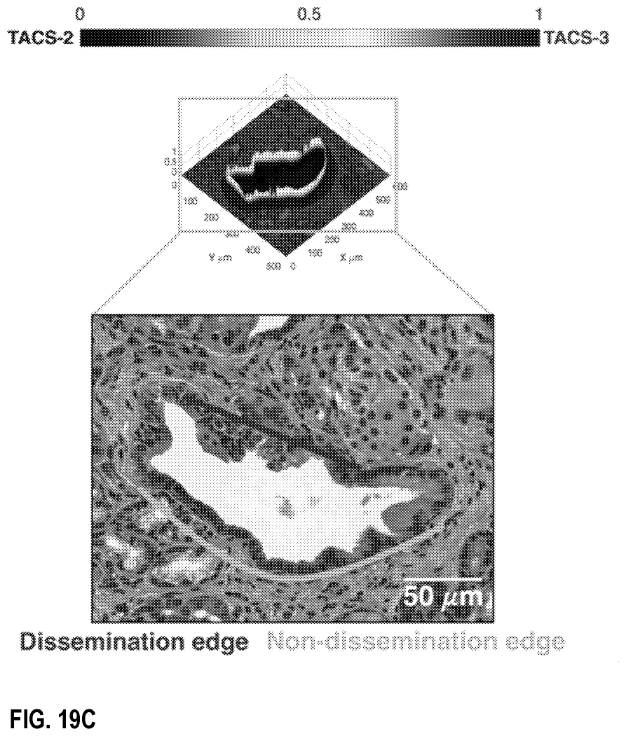

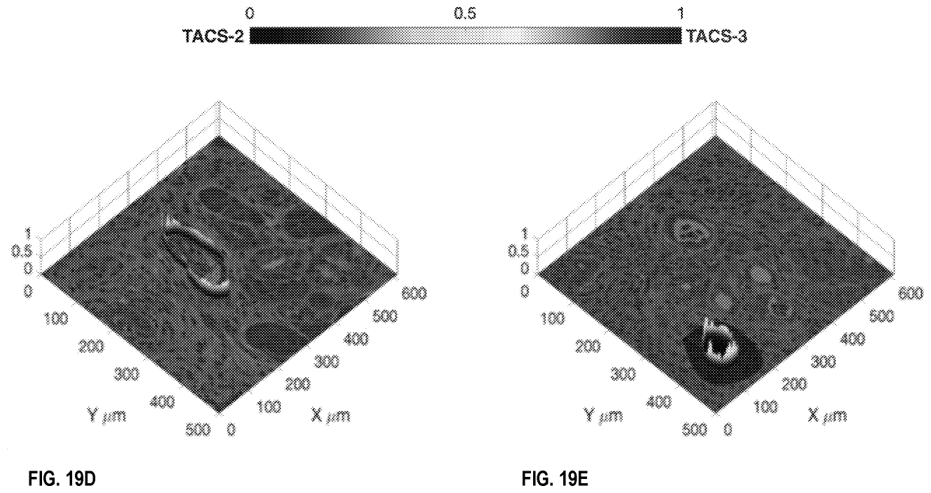

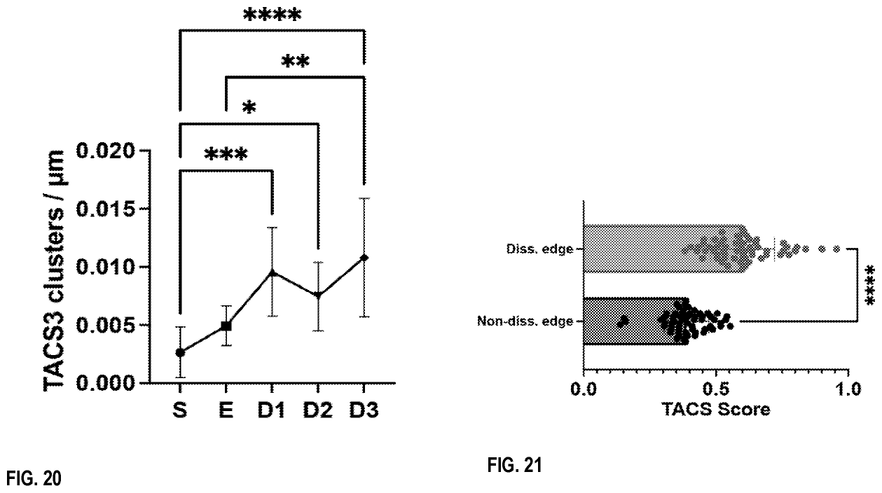

PPM and PS-TACS may effectively detect the transitions to dissemination from PanINs and throughout PDA. As shown in the example flow diagram of FIG. 17, dissemination in PDA can be broadly described as a five-step process with a transitory period marked by aggressive expansion. The ECM initially acts to confine and sustain the cancer cells (Sequestration) but is remodeled as the lesion expands (Expansion) and disease progresses. Matrix is altered (Deformation: D1), leading to invasion from PanINs (Dissemination: D2) or throughout PDA (Dissemination+: D3). FIGS. 18A-18E are PPM images illustrating various stages of PDA disease progression. FIGS. 19A-19E are plots by PS-TACS demonstrating periductal fiber architecture, ranging from TACS-2 to TACS-3. As shown in the example plot of FIG. 20, advanced disease is associated with significantly different (****p<0.0001, Ordinary one-way ANOVA) and increased occurrence of TACS-3 hotspots with post-hoc statistics (*p<0.05, **p<0.01, ***p<0.001, ****p<0.0001). In an example, TACS along the dissemination and non-dissemination edges (see, e.g., FIG. 19C) in PanINs were sampled (e.g., (number of samples) n=30) to elucidate disease heterogeneity within a single duct. As shown in the example plot of FIG. 21, dissemination edges across sampled PanINs may exhibit higher TACS scores with more TACS-3 than non-dissemination edges (****p<0.0001).

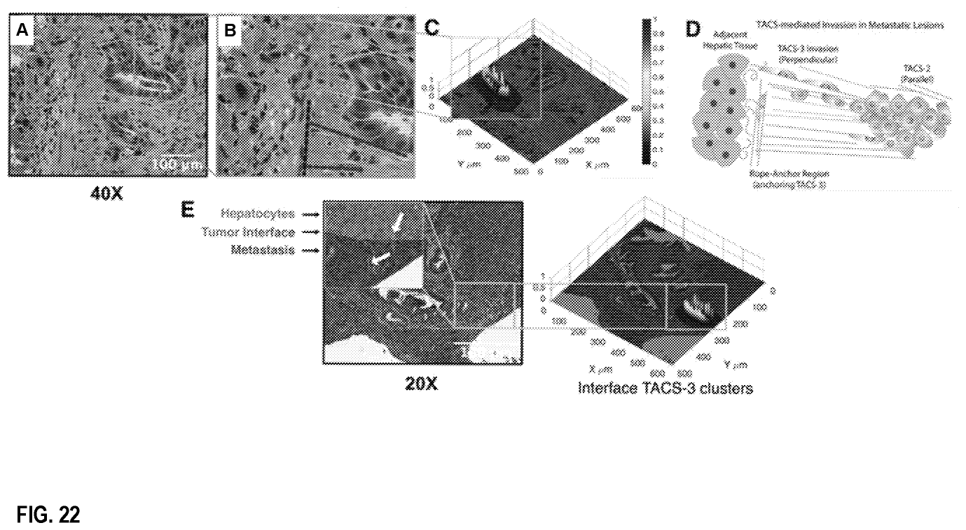

FIG. 22 illustrates an example of ECM architecture in metastatic PDA lesions. As shown in the example of FIG. 22, images A-B, PPM analysis of ECM architecture in PDA liver demonstrates that collagen surrounding carcinoma clusters is largely TACS-2 with notable TACS-3 regions associated with focal and local invasion. At the liver-metastasis interface, wavy fibers are found adjacent to hepatocytes followed by collagen aligned perpendicular to the tumor boundary that are anchoring TACS-3 aligned fibers (vertical dashed line=fibers aligned perpendicular to the tumor boundary; intersecting dashed lines=TACS-3 fibers anchored to a region). In the example of FIG. 22, image C, PS-TACS identifies TACS-3 hotspots in liver metastasis. The rope-anchor model (FIG. 22: D) of cancer invasion comprises bundles of fibers perpendicular to the invasion interface, inserting directly into nearby tissue that brace TACS-3 aligned fiber leading toward hepatic regions. A broader FOV (FIG. 22: E) of a liver micrometastasis shows similar structure, where prominent fiber bundles extend into hepatocytes and create anchors as adhesion points (arrows).

Example methods of performing PPM based characterization of collagen architectures are further described below.

### KPC Mouse Model

Primary and metastatic pancreatic tumors, and normal or associated adjacent normal tissues, may be isolated from the genetically engineered Kras^{G12D/+}; Tp53^{R172H/+}; Pdx1-Cre (KPC) mouse model of pancreatic cancer, which is a highly faithful mouse model for human pancreas cancer that recapitulates the histologic progression of disease, stromal dynamics, metastatic spread and clinical syndromes observed in human disease.

### Tissue Harvest and Staining

Normal and pancreatic and metastatic tumors may be harvested and fixed in formalin and paraffin-embedded to then process as formalin-fixed paraffin-embedded (FFPE) sections that undergo H&E staining for pathologic assessment and imaging.

During the preparation of the specimen slide held by slide holder 114, certain sample preparation techniques may be controlled or selectively omitted to preserve the inherent birefringence of the tissue. For example, antigen retrieval methods that involve applying high heat to the tissue sample can alter or permanently reduce the natural birefringence of the extracellular matrix. Because the polarization microscopy system 100 utilizes this birefringence to generate the optically coded spatial and angular information captured by image sensor 120, processing circuitry 104 may process image data captured specifically from specimen slides prepared without high-heat antigen retrieval. In some examples, if high-heat antigen retrieval is utilized for concurrent staining protocols, processing circuitry 104 may apply a compensatory signal gain during the amplitude correction phase to account for the anticipated reduction in the polarization microscopy signal.

### Second Harmonic Generation Microscopy

FFPE sections may be imaged using a multiphoton laser scanning microscope (e.g., manufactured by Prairie Technologies/Bruker). Collagen fibers in the tissue slides may be verified with SHG using a tunable (e.g., 690-1040 nm), pulsed (e.g., 100fs, 2.5 W) Ti: Sapphire laser (e.g., manufactured by Spectra-Physics). In some examples, the collagen fibers may be imaged at 880 nm (2nd harmonic) in the backward direction through a 20×NA1.0 water-immersion objective lens (e.g., Olympus, XLUMPlanFLN), filtered (e.g., 440/20 bandpass, Chroma), and collected through a multi-alkali PMT (e.g., manufactured by Hamamatsu).

### PPM Microscope and Image Acquisition

In some examples, polarization microscopy (e.g., PPM) system 100 of FIG. 1B may be implemented on an Olympus BX50 microscope body (e.g., manufactured by EVIDENT, Waltham, MA) illuminated by a halogen lamphouse (e.g., U-LH100-3, 100 W, EVIDENT) and with a white light filter. Light subsequently passes through polarization state generator 110 (e.g., polychromatic polarization state generator), which may comprise a polarizer, a Z-cut quartz crystal, and a strain-free condenser with the polarizer removed (e.g., U-POC-2, EVIDENT). Tissue samples may be optically visualized by 10×, 20×, and 40× objectives for polarized light (e.g., using UPLFLN10XP-2, NA: 0.3, WD: 10 mm; UPLFLN20XP-2, NA: 0.5, WD: 2.1 mm; UPLFLN40XP-2, NA: 0.75, WD: 0.51 mm; EVIDENT). In some examples, polarization state analyzer 118 is orthogonally placed with respect to polarization state generator 110. Images may be detected by image sensor 120, which in some examples, may be a charge-coupled device (CCD) camera (e.g., Micropublisher 6 CCD camera having 16 mm diagonal FOV at 6 megapixels, manufactured by Teledyne Photometrics). The scope may be calibrated with a calibration slide (e.g., FIG. 3) imprinted with birefringent patterns (e.g., manufactured by Kazansky lab, University of Southampton) with the slow axis perpendicular to the FOV. In some examples, the calibration slide contains birefringent patterns. The star pattern may have an overall diameter (d) of 60 μm and may comprise three distinct line widths at d=60 μm, d=40 μm and d=20 μm with a maximum line width of 3 μm at d=60 μm. The perpendicular linear grating patterns may comprise 3 distinct clusters with increased line densities of 350, 550, and 1050 lp/mm. In some examples, images may be digitally acquired using an automated microscopy tool (e.g., software executed by processing circuitry 104), such as μManager, an open-source software package for controlling automated microscopes, with a calibrated white balance prior to image acquisition. Exposure may be adjusted on an image-to-image basis to prevent sensor saturation.

### Collagen Segmentation

In some examples, feature segmentation (e.g., Matrix segmentation) performed by pixel classification with a feature segmentation tool (e.g., software executed by processing circuitry 104), such as QuPath. For example, fibers in PPM images from pancreata and liver may be labeled. Non-collagen components may be labeled as background. The Random Trees algorithm may be applied to the labeled images to train an ML classifier for each tissue type, resulting in a classification map generated by the classifier. The classification map may then be transferred as a binary mask to an image processing tool (e.g., software executed by processing circuitry 104), such as FIJI, an open-source image processing package. The resulting segmented RGB collagen-only image may be acquired by masking the original PPM image with the binary mask.

### PPM Image Kalibrator (PIKL)

PIKL is a calibration tool developed in MATLAB to facilitate downstream TACS analysis of PPM images. Calibration using PIKL, or any other functionally similar calibration tool, may be used to correct for both phase and magnitude of the hue. H&E staining shifts the hue of collagen towards magenta to an inconsistent degree, which inherently affects any hue-based angle measurements. This inconsistency happens both across samples and within samples (different regions). Correcting the phase shift removes session-to-session biases that can happen in real-world applications, including alterations to the polarization state generator to and variations in ambient lighting. Calculating and correcting for the phase shift effectively aligns the angle axis [0, pi] to the hue axis [0, 255], making it mathematically convenient for obtaining a curve with least squares regression, which is subsequently used to evaluate angles given any arbitrary hue values. Thus, a reference angle may be provided as guided by PIKL, e.g., per image, to account for that hue shift. PIKL may also correct the magnitude of the hue due to the detector's non-uniform spectral response, as different sensors will intrinsically have different spectral responses. Therefore, PIKL may provide a calibration step that provides tolerance to user errors and variance in experimental methods and instruments and to allow quick adaptation of PPM with different detector configurations.

To obtain the hue correction curve (HCC), a normalization curve may be used that allows continuous sampling of the HCC with infinite angular resolution. In some examples, the hue channel of the calibration slide image is filtered with Gaussian (r=1) and median filter (r=2) to remove optical artifacts due to the roughness in the pattern introduced in the pattern fabrication process. Gaussian filtering with a radius of 2 (e.g., using FIJI) may be applied to smooth out textural inconsistencies during pattern fabrication and chromatic aberration introduced by microscope objectives. Modified Akima interpolation (MAI) may be applied to the (e.g., 16) peaks detected in the histogram corresponding to (e.g., 16) angles of the radial birefringent pattern in the calibration slide. In some examples, the HCC is the envelope of the hue histogram extracted by MAI as a function of intensity in the hue channel, and it is applied to the segmented images for hue-based analysis. To correct the hue histogram value profile as a function of directions, the techniques of this disclosure may include sampling the HCC of an input angle and obtaining a normalization multiplier. The histogram frequency of an input angle may then be normalized to reflect its actual frequency. A phase shift may be present due to the mismatch between the true angle and the calculated angle based on the hue histogram. The calibration slide image can be converted to hue-saturation-value (HSV) color space. Since the calibration pattern images in the HSV color space show angular dependence manifested by distinct pixel histogram frequencies, an uncorrected hue histogram frequency profile as a function of directions may result in quantitative bias. Interference in white balance (WB) from internal and external sources may also introduce a hue shift and thus should be corrected. PIKL may be used to calculate and apply the corrective phase shift (φ) by using the perpendicular birefringent pattern from the calibration slide. In some examples, the true angle is accordingly the circular shift of the uncorrected hue angle by φ. To obtain φ, the line patterns from the calibration slide may be selected in FIJI as RGB images and transformed into HSV color space. The global maximum of the peak in the hue histogram may be automatically detected in PIKL, with each value on the x-axis representing an angle from 0 to π/2. Thus, the peak location may represent the measured angle. In some examples, the corrective phase shift, φ, is simply the difference between the measured angle and the ground truth. To enable correction of phase shift for all angles, hue values from the star patterns can be measured, and a least-square fit of the values produces a 1st-degree polynomial that can be infinitely sampled. PIKL may streamline the hue shift correction of measured angles in a PPM image. In some examples, a graphical user interface (GUI) may be used to prompt a user to select a collagen fiber as the calibration line. PIKL may then automatically calculate the drawn angle, retrieve φ, and apply the correction.

In addition to employing a physical calibration slide, the phase correction or the amplitude correction may be determined and applied using alternative techniques of the disclosure. For example, processing circuitry 104 may apply a predefined correction profile stored in memory 106, wherein the predefined correction profile corresponds to known spectral response variations of an image sensor used to capture the image data. Processing circuitry 104 may also apply algorithmic estimations to correct hue distortion or amplitude distortion. Such algorithmic estimations include extracting baseline hue values from regions of the image data known to lack birefringent features and applying a localized shift to the optically coded spatial and angular information. In some examples, machine learning models trained on paired datasets of distorted and calibrated polarization microscopy images may be executed by processing circuitry 104 to predict and apply the phase correction or the amplitude correction directly to the received image data without real-time physical calibration targets.

### Training the Machine Learning Classifier

In some examples, images may be selected and loaded individually in QuPath to train the ML classifier. Collagen, referenced by SHG, may be labeled accordingly using a brush tool and saved as annotations. During the labeling process, collagen fibers may be randomly selected across the image to maintain an optimal and unbiased representation of all possible fiber configurations. All non-collagen components in the images may be labeled as background, e.g., with extra attention paid to small, non-collagen spaces in order to enhance separability between smaller fiber bundles. In some examples, starting with (e.g., 100) annotations each for collagen fibers and background, respectively, the pixel classification tool may be applied to train the random forest classifier with a maximum number of (e.g., 50) trees and maximum depth of (e.g., 25), using full image resolution. Of note, annotations can vary in the number of pixels, and each pixel may be considered an individual instance. After training, the ML classifier may be applied to the whole image. Quality check may be conducted to ensure optimal collagen fiber segmentation. This process may be iterated until collagen segmentation are satisfactory based on human examination in reference to SHG.

### TACS Characterization

PS-TACS is a pixel-wise analysis tool developed in MATLAB to streamline TACS characterization for PPM images. In some examples, PS-TACS is fully integrated with PIKL for automated corrections and takes input from a reference image and a segmented image from QuPath. Users may be prompted to select a pancreas duct of interest (ROI₁) and the associated stromal collagen fibers (ROI₂). PS-TACS may subsequently calculate the vectors (\vec{n}) orthogonal to the contour pixel (C) of ROI₁. For each intersecting fiber pixel (F) possessing a direction \vec{p} with \vec{n}, the dot product may be calculated to obtain the angle between the fiber pixel and the contour normal vector. Each pixel may be weighted on a zero-mean Gaussian curve, G (x, σ),

$$G ( x , σ ) = \frac{1}{σ \sqrt{2 π}} {e}^{- \frac{{x}^{2}}{2 {σ}^{2}}}$$

while 3σ falling on the mean of minimum contour distance between ROIs (ΔD) such that close fibers have larger weights and distant fibers have smaller weights. The weighted angles (A_{w}) may be expressed as

$${A}_{w} = \frac{1}{n} {\sum }_{j = 1}^{n} ℒ ( \frac{{\vec{n}}_{j} \cdot {\vec{p}}_{j}}{\vert {\vec{n}}_{j} \vert \cdot \vert {\vec{p}}_{j} \vert} ) G ( \sqrt{{( {x}_{c} - {x}_{f , j} )}^{2} + {( {y}_{c} - {y}_{f , j} )}^{2}} , \frac{1}{m} {\sum }_{k = 1}^{m} Δ {D}_{k} )$$

The TACS status(S) of a k-pixel contour may be determined by the transform L(α) of weighted angles α, where α∈[0° 180°]

$$S = \frac{1}{n} {\sum }_{n}^{k} ℒ ( {A}_{w , k} )$$

$$and$$

$$ℒ ( α ) = \{ \begin{matrix}- 1 & if α > a ° and α < 180 - a ° \\ 1 & if α < a ° or α > 180 - a °\end{matrix}$$

In the transformation function above, the variable a represents a tunable angular threshold utilized by processing circuitry 104 to distinguish between different matrix fiber alignments. For example, the tunable angular threshold may define a mathematical boundary between fibers considered parallel to the contour of the first region of interest and fibers considered perpendicular to the contour. Processing circuitry 104 may set this angular threshold dynamically, or it may adjust the angular threshold based on user input received via user interface 107 to fine-tune the strictness of the binary classification between the distinct collagen signatures.

### Collagen Metrics

Collagen length, width, and straightness can be extracted by CT-FIRE. Periductal collagen fibers for the pancreas and tumor-associated collagen fibers in liver metastases may be manually selected as ROIs. ROIs may be imported into CT-FIRE in batch processing mode for parallelized collagen metrics computation.

Information that characterizes the one or more birefringent features encompasses a wide variety of metrics and data types generated from the corrected spatial and angular information. While specific examples include TACS regression scores and collagen structural metrics such as length and width, processing circuitry 104 may generate other characterizing information. This characterizing information may include, but is not limited to, density measurements, localized alignment scoring, feature boundary mapping, structural integrity estimations, and algorithmic classification labels. Processing circuitry 104 may apply clustering algorithms to the corrected spatial and angular information to group birefringent features into spatial distribution maps. In some examples, processing circuitry 104 may output quantification metrics that define a total area, volume, and/or relative proportion of the birefringent features within a targeted ROI.

Based on the above-described example methods of performing PPM based characterization of collagen architectures, PPM reveals dense collagen architectures in autochthonous PDA with inherent angular information. In some examples, polarization microscopy (e.g., PPM) system 100 is calibrated with the calibration slide of FIG. 3. PPM utilizes a few fundamental optics beyond a traditional polarization microscopy setup (e.g., see FIG. 1B). In some examples, illumination from light source 108 passes through the polarization state generator 110 (e.g., made from z-cut quartz (SiO₂)), rotating the polarization in a wavelength-dependent manner and resulting in dispersed polarization states. Fibrillar ECM is birefringent and may therefore process a slow axis orientation and retardance. A color component of the beam transmitted through the specimen may be extinguished by the inverse polarization rotation of the analyzer 118 if the incoming polarization states remain unaltered by the specimen. In some examples, the hue of a matrix fiber such as collagen, hence, depends on the orientation of the fiber with respect to the slow axis orientation. To confirm the specificity of collagen contrast in PPM, SHG and PPM imaging can be performed on the same section. As shown in FIG. 4, SHG imaging of PDA exhibits a strong collagen signal, which is largely absent and unspecific with bright-field microscopy. However, PPM simultaneously renders detailed collagen fiber architecture and analog orientation information for direct observation by eye or digital acquisition. Thus, for pathologic assessment, PPM involves low initial setup (e.g., 30-50X cheaper than SHG, and even less if adding it to an existing light microscope), maintenance, and sample preparation, prompting unique and tangible opportunities in early diagnosis from H&E stained samples.

Based on the above-described example methods of performing PPM based characterization of collagen architectures, ML-based collagen segmentation and PIKL may produce accurate hue-based collagen metrics. Collagen segmentation in H&E images acquired by PPM can be inherently challenging due to similarities in the hue produced by PPM and the eosin stain and heterogeneity in staining density. The intensity of the Newton colors produced by PPM may be insufficient for intensity thresholds, and methods based on color thresholding in HSV and LAB color spaces may be biased toward pink/magenta due to the presence of eosin. However, ML-based pixel classification provides high tunability, specificity, and throughput for fiber segmentation at scale. Following the example workflow of FIG. 5, collagen fibers may be labeled, and a random forest classifier may be trained to classify collagen fibers in all PPM images. The accuracy and generalizability of the classifier may be iteratively tuned by manual labeling of collagen fibers across various image samples. The ML classifier has been proven highly effective in fiber segmentation (e.g., as shown in FIGS. 6A-6C).

The feature segmentation pipeline utilized by computing device 102 may be modular and software-agnostic. While machine learning classifiers or specific digital pathology platforms may be employed, the techniques of the disclosure are not limited to any single segmentation algorithm or software tool. Because the downstream phase and amplitude corrections applied by processing circuitry 104 utilize a validated binary mask or similar segmented image data as an input, processing circuitry 104 can readily integrate substitute segmentation algorithms, third-party software tools, or updated neural network models as they become available. This modularity enables the polarization microscopy system 100 to adapt to new computational technologies without a fundamental redesign of the optical analysis workflow.

In some examples, image sensor 120 is a CCD sensor (e.g., CCD camera) that works by capturing photons and converting them into electrons, which is known as the photoelectric effect. However, the quantum efficiency of such conversion as a function of wavelengths may not be uniform, which can result in biased intensity undesirable for quantification. In addition, the hue histogram generated by the linear patterns (FIG. 7A) and its corresponding hue value (FIG. 7C) may be mismatched by a phase shift. Applying a phase correction may simplify angular calculation and least squares regression that mandates continuity of hue values across the 8-bit histogram. Thus, to standardize PPM image analysis pipeline and facilitate pathologic assessment using PPM-based image evaluation, a calibration tool (e.g., PIKL) may be used to correct for non-uniform spectral response and applies the phase correction. Using the hue channel image (FIG. 2G), Gaussian filtering may be applied to smooth out textural inconsistencies and chromatic aberration introduced by microscope objectives. A uniform ROI may be selected inside the star pattern so that the area of each wedge is equivalent to avoid bias in hue histogram frequencies as a function of directions. Accordingly, a sensor with uniform spectral response may produce (e.g., 16) peaks with the same amplitude in the hue histogram, with each peak accounting for one of the (e.g., 16) directions.

In practice, the foregoing methods demonstrate that the amplitudes of the peaks are not uniform (FIG. 7D) due to the non-uniform spectral response of the image sensor. By importing the hue histogram from FIJI to PIKL, the peaks corresponding to each direction may be extracted. The HCC (FIG. 8) and the phase correction may be obtained as outputs of the image calibration process in PIKL. The resulting polar plots (FIGS. 9A-9B) display the angle measurements of the star and linear patterns before and after correction. After correction, the star pattern may exhibit (e.g., 16) distinct peaks (due to amplitude correction) and matching angles (due to phase correction). In some examples, the linear pattern may produce one peak at 90° after correction, which matches the ground-truth angle of the perpendicular line pattern.

To examine the feasibility of angular measurement by hue values, a polynomial fit may be applied to the hue histogram intensities of the star pattern as a function of (e.g., 16) directions. The hue values may reflect the angles accurately (e.g., R2=0.9831). Therefore, the angle of a pixel can be found by evaluating its hue value with the polynomial, which is continuous throughout the domain. Angle measurement in PDA images can be tested with two different ROIs, e.g., a small ROI containing a segment of mostly straight fibers in the same direction (FIG. 11A), which outputs as one dominant peak in the polar histogram (FIG. 12A), and a large ROI containing an entire duct with more heterogeneous collagen features (e.g., FIG. 11B) that produce multiple peaks with different angles and magnitude (FIG. 12B). Based on the methods and testing described above, it has been shown that the computational analysis platform may accurately and directly obtain quantitative information about ECM architecture in thin tumor sections.

Quantitative analysis of PPM images may capture collagen architectures associated with PDA progression. TACS architectures have been observed in multiple tumor types where they serve to promote disease progression to metastasis and are particularly strong in PDA. Furthermore, these ECM patterns promote both early dissemination in PDA as well as continual avenues for focal and local invasion and access to blood vessels in both early and late-stage disease. As such, approaches to rapidly visualize and quantitively evaluate these patterns in thin archival sections have wide value in both studies of the tumor microenvironment and pathologic assessment. Notably, PPM is effective for both wet lab and clinical environments due to its capability of rapidly producing orientation-coded collagen contrast with standard H&E slides. Therefore, with the herein described PPM image processing platforms, PPM-based techniques may demonstrate faithful analysis of collagen architectures in pancreatic cancer as a function of disease stage (i.e., Normal to PanINs to PDA). This has been observed using the KPC genetically engineered mouse model of human PDA (FIG. 13). Normal pancreatic samples have minimal periductal birefringence (FIG. 13: A, D, G), suggesting that the ECM normal ducts may not be composed of dense fibrillar structures. PanINs, on the other hand, may present with strong ECM birefringence, consistent with the known desmoplasia chronologically of PDA with increased collagen density, fiber length, and width as the disease advances (FIG. 14). In the example of FIGS. 13-14, the majority of collagen surrounding PanINs is in the TACS-2 configuration with notable focal TACS-3 regions, consistent with recent findings examining collagen architecture during early dissemination surrounding PanINs, demonstrating that PPM can identify structural information that is consistent with collagen imaging with SHG. Examination of moderately to poorly differentiated PDA (FIG. 13: C, F, I) demonstrates long collagen fiber bundles with associated streams of cells and heterogeneity of TACS-3 aligned fiber orientations in different regions of the tumor section image. At this stage, carcinoma cells have infiltrated and re-distributed throughout the ECM, where they use TACS-3 aligned fibers that remain throughout the tumor volume to move through the tumor mass and toward blood vessels. Thus, in thin archival samples, PPM can capture target ECM architectures that have been implicated in both early and late-stage PDA progression.

PS-TACS may compute pixel-wise spatial TACS regression. To develop a computational framework to identify and quantify TACS near or around areas of interest (e.g., surrounding PanINs, carcinoma cell clusters, lines of tumor cells, etc.), a quantitative analysis tool for PPM images may be used to compute a pixel-wise TACS score. Within early and late disease, collagen architectures are seldom purely TACS-2 or TACS-3 within a given ROI. For instance, while ductal structures are TACS-2 dominant, they may often include TACS-2 and -3 architectures (FIG. 16: A). In this context, it may be of pivotal interest to define spatial differences in TACS as these ECM orientations influence early dissemination behaviors. PS-TACS may be used to define an ROI and measure TACS, pixel-wise, relative to the ROI feature (FIG. 16: B). In the PanIN context, the duct and ECM may be defined as first and second ROIs (e.g., ROI₁ and ROI₂) based on user selection (FIG. 16: C, D). This step may establish the structure to be analyzed and the ECM fiber features to be included in the TACS calculation. PS-TACS may accordingly be border-dependent and border-constrained. For a contour pixel C (x, y) along the border of ROI₁, angles between its normal vector \vec{n} and their intersecting fiber tangent vectors \vec{p}_{c} may be computed (FIG. 16: B). Angles may be subsequently transformed into binary TACS scores according to a tunable angular threshold. The distances from the fiber pixel P to contour pixel C may be evaluated on a dynamic Gaussian curve tuned to represent the mean distance between ROI₁ and ROI₂, such that the standard deviation

$$σ = \frac{Δ {D}_{m}}{3} .$$

This step may ensure a gradual falloff of weights, and fibers further from the border contribute less to the TACS calculation. Pixel TACS regression may be obtained by taking the weighted average. A binary classification overlay of the border may be calculated to represent the overall TACS class, where some border pixels are TACS-2 and some pixels are TACS-3 (FIG. 16: C, D). With this analysis, it is evident that the large duct (FIG. 16: C) is mostly classified as TACS-2 with TACS-3 hotspots, while the small duct (highlighted in FIG. 16: D) shows more robust TACS-3 due to the dense fiber bundles running to the duct and encapsulating the rest of the borders in a TACS-2 fashion. PS-TACS may additionally compute and visualize the TACS regression (i.e., TACS score) on a linear scale from 0 to 1, where TACS-2 and TACS-3 features are represented along the defined border (FIG. 16: E, F), allowing TACS calculation for virtually any ROI and enabling further analysis on collagen-mediated cancer dissemination.

PPM with PS-TACS may effectively detect the transitions to dissemination from PanINs and throughout PDA. PS-TACS may be applied to evaluate pancreatic samples at differing stages of disease to better understand development of early dissemination architectures in pancreatic cancer. As disease advances, there may be a defined histologic progression in PDA accompanied by a stromal evolution. KPC mice may recapitulate this histologic progression. As this evolution is observed, it may be possible to roughly categorize the physical processes into five phases (FIG. 17) with distinct numbers of TACS clusters. During Sequestration(S), fibrosis constrains the ductal structure (FIG. 18A), akin to early findings in breast carcinomas. During Expansion (E), as PanIN abnormality increases, there is an outward expansion force exerted on the ECM. As disease progresses, replication of aberrant epithelial cells continues and further crowds the ductal space, and carcinoma in situ develops with outward force on the constraining matrix. The space surrounding the duct may primarily include TACS-2 fibers (FIG. 18B). TACS-3 clusters exist but are often perpendicular fibers slightly further away from the duct boundary. However, overall, heterogeneous matrix structures are more present and there are more TACS-3 architectures emerging during this phase (FIG. 18B). As this progresses, local deformation, remodeling, and ECM deposition are more prominent and, the Deformation (DI) stage emerges where TACS-3 clusters are often found and formed by perpendicularly connecting into the periphery of warped ductal boundary where invasion can begin (FIG. 18C). Moving through the fibrotic barrier via aligned fibers marks the initiation of the early dissemination and this process continues into more robust invasion from well-differentiated PDA, e.g. Dissemination (D2) phase from PanINs or Dissemination+ (D3) phase from differentiated PDA, where cells in the tumor are now predominantly moving along aligned fibers that are TACS-3 structures that remain throughout the tumor (FIGS. 18D-18E). During D2, multifocal TACS-3 bundles are more prominent with connection points of dissemination and provide contact guidance directed dissemination. There can be unifocal or multifocal dissemination and in some cases high variation of disease. To quantitatively capture these features, PS-TACS can be applied to images of diseased pancreata stratified by the physical S.E.D.D.D processes, which allows further evaluation of the number of TACS-3 clusters (FIGS. 19A-19E).

Analysis of early dissemination features normalized by ROI border length in pancreas ducts stratified by their dissemination stage demonstrates that pancreatic ducts during each phase have a varied amount of TACS-3 clusters (FIG. 20; p<0.0001, one-way ANOVA), suggesting that TACS-3 may present in pancreas cancer as a differentiating marker that demarcates the degree of potential dissemination. Post hoc analysis shows that ECM remodeling primarily occurs during the Deformation phase and continues as disease progresses.

FIG. 19C illustrates an example of surveying PanINs with disease variation along the ductal epithelium and sampling for PS-TACS scores along dissemination edges and non-dissemination edges. This example demonstrates that dissemination edges may have perpendicular collagen fibers including more TACS-3 than non-dissemination edges, which may signify that early dissemination not only necessitates ECM remodeling, but it also prefers a TACS-3 architecture demonstrated by PPM and PS-TACS.

While a great deal of investigation is reporting on stromal architectures in primary tumors, much less is known about TMEs in metastatic lesions. In PDA, the liver is the most common metastatic site. As such, the techniques of this disclosure have been applied to evaluate metastatic PDA in liver from KPC mice. Within metastatic tumors with well-differentiated disease features, collagen surrounding ductal structures is again seen as largely TACS-2 with additional regions of TACS-3, which are associated with features of focal invasion (FIG. 22: A). Aligned ECM is also observed throughout the tumor with associated cell interactions. Interestingly, collagen fibers at the invasion front into adjacent hepatic tissue present with aligned TACS3 fiber structures into adjacent normal hepatic regions that provide an avenue for local invasion (FIG. 22: A, B). Examining focal invasion into adjacent tissue shows large fiber bundles that cut through the PDA-liver interface providing an avenue for contact guidance (FIG. 22: B). In addition, there are large fiber bundles leading to the PDA-liver interface that are immediately adjoined to long perpendicular fibers (FIG. 22: B, C). The aligned fibers appear to interface to a perpendicular fiber network that is a barrier that is adjacent to a looser ECM associated with the hepatic tissue near the boundary (FIG. 22: B, C), likely providing an anchor for the aligned fibers guiding invasive carcinoma cells through contact guidance to the interface, suggesting that this connection may be a precursor that later results in frank local invasion into adjacent tissue. Indeed, these “rope-anchor” architectures, bracing TACS fibers are robustly found along the tumor-liver boundary in micrometastatic lesions (FIG. 22: D). For instance, examination of the liver boundary and the region surrounding ductal structures with PS-TACS demonstrates that multiple TACS-3 clusters in addition to the parallel anchor fibers (FIG. 22: E). Collectively, the findings suggest that desmoplasia in metastatic PDA promotes further invasion, and likely additional points of dissemination, in a causal manner that is facilitated by the interplay between rope fibers and anchor fibers that promote directed invasion.

This disclosure describes and demonstrates advantages of polarization microscopy techniques, such as PPM, paired with PIKL and PS-TACS for downstream image calibration and TACS analysis in order to examine ECM architecture in PDA. Overall, PPM is an apt substitute for, and may improve upon, SHG imaging for ECM fibers in thin 2D sections, as demonstrated through direct comparison, delivering analog angular information unique to PPM directly through the eyepiece. PS-TACS, fully integrated with PIKL, may streamline instrument calibration and deliver tunable, spatially resolved TACS computation. The resolution may be limited by the dynamic range and resolution of the image, and an improved imaging sensor can drastically improve results further.

Desmoplasia with robust ECM deposition, and in particular high levels of collagen, is one of the hallmarks of PDA. Along these lines, it is defined that collagen fiber alignment helps drive disease progression to metastasis. Using PPM and PS-TACS, the techniques of this disclosure are able to evaluate histology samples at varying stages and define ECM architectures during the multistep process that progresses to early dissemination explicitly guided by the formation and evolution of TACS-3 and architectures that continue to drive dissemination in later-stage disease. Although fibrosis surrounding early PanINs may constrain cancer dissemination, similar to observations in breast cancer, in more advanced PanINs and frank PDA, it instead promotes dissemination. Veritably, examination of disease heterogeneity along the dissemination edge of pancreatic ducts or carcinoma cluster further confirms that more advanced disease is associated with TACS-3 matrix architectures. Likewise, a clear distinction between the number of TACS-3 clusters in LG-PanIN, HG-PanIN, and PDA suggests that collagen architecture can be used as a physical diagnostic marker. For metastatic PDA in the liver, including micrometastasis of the liver, the techniques of this disclosure can be used to demonstrate evidence of TACS-mediated focal and local invasion. By examining this tumor-normal adjacent interface, it is possible to observe that the TACS-3 fibers are anchored at the boundary, i.e., facilitated by the rope-anchor mechanism, suggesting this may be a precursor to local spread. PS-TACS further showed the formation of TACS-3 clusters at the invasion front. Thus, polarization microscopy (e.g., PPM) based techniques of this disclosure demonstrate potential in characterizing the ECM in sections from both primary and metastatic PDA that can serve as a robust platform for TME research and provide great clinical diagnostic value.

Based upon the above discussion and illustrations, it is recognized that various modifications and changes may be made to the disclosed technology in a manner that does not necessarily require strict adherence to the examples and applications illustrated and described herein. Such modifications do not depart from the true spirit and scope of various aspects of the disclosure, including aspects set forth in the claims.

In at least one example, the functions described may be implemented in hardware, software, firmware, or any combination thereof. If implemented in software, the functions may be stored on or transmitted over as at least one instruction or code on a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable data storage media can include RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Combinations of the above should also be included within the scope of computer-readable media.

Instructions may be executed by one or more processors, such as one or more DSPs, general purpose microprocessors, ASICs, FPGAs, CPLDs, or other equivalent integrated or discrete logic circuitry. Accordingly, the term “processor” or “processing circuitry” as used herein may refer to any of the foregoing structure or any other structure suitable for implementation of the techniques described herein. Also, the techniques could be fully implemented in at least one circuit or logic element.

Any of the above-mentioned “processors,” and/or devices incorporating any of the above-mentioned processors or processing circuitry, may, in some instances, be referred to herein as, for example, “computers,” “computer devices,” “computing devices,” “hardware computing devices,” “hardware processors,” “processing units,” “processing circuitry,” etc. Computing devices of the above examples may generally (but not necessarily) be controlled and/or coordinated by operating system software, such as Mac OS, IOS, Android, Chrome OS, Windows OS (e.g., Windows XP, Windows Vista, Windows 7, Windows 8, Windows 10, Windows 11, Windows Server, etc.), Windows CE, Unix, Linux, SunOS, Solaris, Blackberry OS, VxWorks, or other suitable operating systems. In some examples, the computing devices may be controlled by a proprietary operating system. Conventional operating systems control and schedule computer processes for execution, perform memory management, provide file system, networking, I/O services, and provide UI functionality, such as GUI functionality, among other things.

The techniques of this disclosure may be implemented in a wide variety of devices or apparatuses, including an integrated circuit (IC) or a set of ICs (e.g., a chip set). Various components, modules, or units are described in this disclosure to emphasize functional aspects of devices configured to perform the disclosed techniques, but do not necessarily require realization by different hardware units.

This disclosure includes the following non-limiting examples.
Example 1. A system for characterizing birefringent features, the system comprising processing circuitry configured to: receive image data that includes optically coded spatial and angular information about one or more birefringent features; apply at least one of a phase correction or an amplitude correction to resolve at least one of a hue distortion or an amplitude distortion affecting the optically coded spatial and angular information to generate corrected spatial and angular information; generate, based on the corrected spatial and angular information, information that characterizes the one or more birefringent features; and output the information that characterizes the one or more birefringent features.
Example 2. The system of example 1, wherein the image data is collected by a polarization microscopy system.
Example 3. The system of any of examples 1 and 2, wherein the processing circuitry is configured to perform feature segmentation on the image data to generate segmented image data, and wherein to apply the at least one of the phase correction or the amplitude correction, the processing circuitry is configured to apply the at least one of the phase correction or the amplitude correction to the segmented image data to resolve the at least one of the hue distortion or the amplitude distortion affecting the optically coded spatial and angular information to generate the corrected spatial and angular information.
Example 4. The system of any of examples 1-3, wherein the processing circuitry is configured to perform a pixel-wise analysis on the image data to extract spatial and angular information about the one or more birefringent features from the corrected spatial and angular information to generate extracted spatial and angular information, and wherein to generate the information that characterizes the one or more birefringent features, the processing circuitry is configured to generate, based on the extracted spatial and angular information, the information that characterizes the one or more birefringent features.
Example 5. The system of example 4, wherein the processing circuitry is configured to perform feature segmentation on the image data to generate segmented image data, and wherein to perform the pixel-wise analysis on the image data to extract the spatial and angular information about the one or more birefringent features from the corrected spatial and angular information, the processing circuitry is configured to perform the pixel-wise analysis on the segmented image data.
Example 6. The system of any of examples 1-5, wherein the processing circuitry is further configured to output the information that characterizes the one or more birefringent features via a user interface.
Example 7. The system of any of examples 1-6, wherein the processing circuitry is further configured to transmit the information that characterizes the one or more birefringent features to another device.
Example 8. The system of any of examples 1-7, wherein the one or more birefringent features include tumor-associated collagen signatures (TACS).
Example 9. The system of example 8, wherein to generate the information that characterizes the one or more birefringent features, the processing circuitry is configured to determine at least one of a quantification or a spatial distribution of one or more types of TACS present in the image data.
Example 10. The system of example 9, wherein to generate the information that characterizes the one or more birefringent features, the processing circuitry is further configured to generate diagnostic information based on the at least one of the quantification or the spatial distribution of the one or more types of TACS present in the image data.
Example 11. The system of any of examples 1-10, wherein the processing circuitry is further configured to receive a user input indicating a region of interest in the image data, and wherein to generate the information that characterizes the one or more birefringent features, the processing circuitry is further configured to limit the information that characterizes the one or more birefringent features to the region of interest.
Example 12. The system of any of examples 1-11, further comprising a polarization microscopy system that collects the image data.
Example 13. The system of example 12, wherein the polarization microscopy system includes the processing circuitry.
Example 14. The system of any of examples 1-13, further comprising a polarization microscopy attachment configured to be coupled to a microscope to form a polarization microscopy system that collects the image data.
Example 15. The system of example 14, wherein the polarization microscopy attachment includes the processing circuitry.
Example 16. The system of any of examples 1-15, wherein a polarization microscopy system collects the image data, and the polarization microscopy system is a polychromatic polarization microscopy (PPM) system.
Example 17. A method of characterizing birefringent features, the method comprising: receiving, by processing circuitry, image data that includes optically coded spatial and angular information about one or more birefringent features; applying, by the processing circuitry, at least one of a phase correction or an amplitude correction to resolve at least one of a hue distortion or an amplitude distortion affecting the optically coded spatial and angular information to generate corrected spatial and angular information; generating, by the processing circuitry, based on the corrected spatial and angular information, information that characterizes the one or more birefringent features; and outputting, by the processing circuitry, the information that characterizes the one or more birefringent features.
Example 18. The method of example 17, wherein the image data is collected by a polarization microscopy system.
Example 19. The method of any of examples 17 and 18, further comprising performing, by the processing circuitry, feature segmentation on the image data to generate segmented image data, and wherein applying, by the processing circuitry, the at least one of the phase correction or the amplitude correction comprises applying, by the processing circuitry, the at least one of the phase correction or the amplitude correction to the segmented image data to resolve the at least one of the hue distortion or the amplitude distortion affecting the optically coded spatial and angular information to generate the corrected spatial and angular information.
Example 20. The method of any of examples 17-19, further comprising performing, by the processing circuitry, a pixel-wise analysis on the image data to extract spatial and angular information about the one or more birefringent features from the corrected spatial and angular information to generate extracted spatial and angular information, and wherein generating, by the processing circuitry, the information that characterizes the one or more birefringent features comprises generating, by the processing circuitry, the information that characterizes the one or more birefringent features based on the extracted spatial and angular information.
Example 21. The method of example 20, further comprising performing, by the processing circuitry, feature segmentation on the image data to generate segmented image data, and wherein performing, by the processing circuitry, the pixel-wise analysis on the image data to extract the spatial and angular information about the one or more birefringent features from the corrected spatial and angular information comprises performing, by the processing circuitry, the pixel-wise analysis on the segmented image data.
Example 22. The method of any of examples 17-21, further comprising outputting, by the processing circuitry, the information that characterizes the one or more birefringent features via a user interface.
Example 23. The method of any of examples 17-22, further comprising transmitting, by the processing circuitry, the information that characterizes the one or more birefringent features to another device.
Example 24. The method of any of examples 17-23, wherein the one or more birefringent features include tumor-associated collagen signatures (TACS).
Example 25. The method of example 24, wherein generating, by the processing circuitry, the information that characterizes the one or more birefringent features comprises determining, by the processing circuitry, at least one of a quantification or a spatial distribution of one or more types of TACS present in the image data.
Example 26. The method of example 25, wherein generating, by the processing circuitry, the information that characterizes the one or more birefringent features further comprises generating, by the processing circuitry, diagnostic information based on the at least one of the quantification or the spatial distribution of the one or more types of TACS present in the image data.
Example 27. The method of any of examples 17-26, further comprising receiving, by the processing circuitry, a user input indicating a region of interest in the image data, and wherein generating, by the processing circuitry, the information that characterizes the one or more birefringent features comprises limiting the information that characterizes the one or more birefringent features to the region of interest.
Example 28. The method of any of examples 17-27, wherein receiving, by the processing circuitry, the image data comprises receiving the image data from a polychromatic polarization microscopy (PPM) system.
Example 29. A non-transitory computer-readable storage medium having instructions encoded thereon, the instructions configured to cause processing circuitry to: receive image data that includes optically coded spatial and angular information about one or more birefringent features; apply at least one of a phase correction or an amplitude correction to resolve at least one of a hue distortion or an amplitude distortion affecting the optically coded spatial and angular information to generate corrected spatial and angular information; generate, based on the corrected spatial and angular information, information that characterizes the one or more birefringent features; and output the information that characterizes the one or more birefringent features.

Various examples have been described. These and other examples are within the scope of the following claims.

What is claimed is:

1. A system for characterizing birefringent features, the system comprising:
processing circuitry configured to:
receive image data that includes optically coded spatial and angular information about one or more birefringent features;
apply at least one of a phase correction or an amplitude correction to resolve at least one of a hue distortion or an amplitude distortion affecting the optically coded spatial and angular information to generate corrected spatial and angular information;
generate, based on the corrected spatial and angular information,
information that characterizes the one or more birefringent features; and
output the information that characterizes the one or more birefringent features.

2. The system of claim 1, wherein the processing circuitry is configured to perform feature segmentation on the image data to generate segmented image data, and wherein to apply the at least one of the phase correction or the amplitude correction, the processing circuitry is configured to apply the at least one of the phase correction or the amplitude correction to the segmented image data to resolve the at least one of the hue distortion or the amplitude distortion affecting the optically coded spatial and angular information to generate the corrected spatial and angular information.

3. The system of claim 1, wherein the processing circuitry is configured to perform a pixel-wise analysis on the image data to extract spatial and angular information about the one or more birefringent features from the corrected spatial and angular information to generate extracted spatial and angular information, and wherein to generate the information that characterizes the one or more birefringent features, the processing circuitry is configured to generate, based on the extracted spatial and angular information, the information that characterizes the one or more birefringent features.

4. The system of claim 3, wherein the processing circuitry is configured to perform feature segmentation on the image data to generate segmented image data, and wherein to perform the pixel-wise analysis on the image data to extract the spatial and angular information about the one or more birefringent features from the corrected spatial and angular information, the processing circuitry is configured to perform the pixel-wise analysis on the segmented image data.

5. The system of claim 1, wherein the one or more birefringent features include tumor-associated collagen signatures (TACS).

6. The system of claim 5, wherein to generate the information that characterizes the one or more birefringent features, the processing circuitry is configured to determine at least one of a quantification or a spatial distribution of one or more types of TACS present in the image data.

7. The system of claim 6, wherein to generate the information that characterizes the one or more birefringent features, the processing circuitry is further configured to generate diagnostic information based on the at least one of the quantification or the spatial distribution of the one or more types of TACS present in the image data.

8. The system of claim 1, wherein the processing circuitry is further configured to receive a user input indicating a region of interest in the image data, and wherein to generate the information that characterizes the one or more birefringent features, the processing circuitry is further configured to limit the information that characterizes the one or more birefringent features to the region of interest.

9. The system of claim 1, further comprising a polarization microscopy system configured to collect the image data.

10. The system of claim 9, wherein the polarization microscopy system is a polychromatic polarization microscopy (PPM) system.

11. A method of characterizing birefringent features, the method comprising:
receiving, by processing circuitry, image data that includes optically coded spatial and angular information about one or more birefringent features;
applying, by the processing circuitry, at least one of a phase correction or an amplitude correction to resolve at least one of a hue distortion or an amplitude distortion affecting the optically coded spatial and angular information to generate corrected spatial and angular information; and
generating, by the processing circuitry, based on the corrected spatial and angular information, information that characterizes the one or more birefringent features; and
outputting, by the processing circuitry, the information that characterizes the one or more birefringent features.

12. The method of claim 11, further comprising performing, by the processing circuitry, feature segmentation on the image data to generate segmented image data, and wherein applying, by the processing circuitry, the at least one of the phase correction or the amplitude correction comprises applying, by the processing circuitry, the at least one of the phase correction or the amplitude correction to the segmented image data to resolve the at least one of the hue distortion or the amplitude distortion affecting the optically coded spatial and angular information to generate the corrected spatial and angular information.

13. The method of claim 11, further comprising performing, by the processing circuitry, a pixel-wise analysis on the image data to extract spatial and angular information about the one or more birefringent features from the corrected spatial and angular information to generate extracted spatial and angular information, and wherein generating, by the processing circuitry, the information that characterizes the one or more birefringent features comprises generating, by the processing circuitry, based on the extracted spatial and angular information, the information that characterizes the one or more birefringent features.

14. The method of claim 13, further comprising performing, by the processing circuitry, feature segmentation on the image data to generate segmented image data, and wherein performing, by the processing circuitry, the pixel-wise analysis on the image data to extract the spatial and angular information about the one or more birefringent features from the corrected spatial and angular information comprises performing, by the processing circuitry, the pixel-wise analysis on the segmented image data.

15. The method of claim 11, wherein the one or more birefringent features include tumor-associated collagen signatures (TACS).

16. The method of claim 15, wherein generating, by the processing circuitry, the information that characterizes the one or more birefringent features comprises determining, by the processing circuitry, at least one of a quantification or a spatial distribution of one or more types of TACS present in the image data.

17. The method of claim 16, wherein generating, by the processing circuitry, the information that characterizes the one or more birefringent features further comprises generating, by the processing circuitry, diagnostic information based on the at least one of the quantification or the spatial distribution of the one or more types of TACS present in the image data.

18. The method of claim 11, further comprising receiving, by the processing circuitry, a user input indicating a region of interest in the image data, and wherein generating, by the processing circuitry, the information that characterizes the one or more birefringent features comprises limiting the information that characterizes the one or more birefringent features to the region of interest.

19. The method of claim 11, wherein receiving, by the processing circuitry, the image data comprises receiving the image data from a polychromatic polarization microscopy (PPM) system.

20. A non-transitory computer-readable storage medium having instructions encoded thereon, the instructions configured to cause processing circuitry to:
receive image data that includes optically coded spatial and angular information about one or more birefringent features;
apply at least one of a phase correction or an amplitude correction to resolve at least one of a hue distortion or an amplitude distortion affecting the optically coded spatial and angular information to generate corrected spatial and angular information;
generate, based on the corrected spatial and angular information, information that characterizes the one or more birefringent features; and
output the information that characterizes the one or more birefringent features.
